SEQUENCE LISTING (66 sequences)

SEQ ID NO: 1 - DNA, 20 bp, Artificial Sequence
  synthetic oligonucleotide primer
ctcagaaagtgacccgccat

SEQ ID NO: 2 - DNA, 20 bp, Artificial Sequence
  synthetic oligonucleotide primer
cctccatctccaacccccta

SEQ ID NO: 3 - DNA, 20 bp, Artificial Sequence
  synthetic oligonucleotide primer
cgttcaggtctgctgacagt

SEQ ID NO: 4 - DNA, 20 bp, Artificial Sequence
  synthetic oligonucleotide primer
ttttgtgactgccacccact

SEQ ID NO: 5 - DNA, 20 bp, Artificial Sequence
  synthetic oligonucleotide primer
gggctggctcagaagacaat

SEQ ID NO: 6 - DNA, 20 bp, Artificial Sequence
  synthetic oligonucleotide primer
ggagctggatttggggttca

SEQ ID NO: 7 - DNA, 21 bp, Artificial Sequence
  synthetic oligonucleotide primer
ggtctccattgggtcaatgct

SEQ ID NO: 8 - DNA, 20 bp, Artificial Sequence
  synthetic oligonucleotide primer
ggggacttcacgttcacact

SEQ ID NO: 9 - DNA, 20 bp, Artificial Sequence
  synthetic oligonucleotide primer
cctgcctccgggctcacctc

SEQ ID NO: 10 - DNA, 19 bp, Artificial Sequence
  synthetic oligonucleotide primer
agcccggaggcagggcctc

SEQ ID NO: 11 - DNA, 26 bp, Artificial Sequence
  synthetic oligonucleotide primer
caccgtctgaggcacgttggccgtct

SEQ ID NO: 12 - DNA, 26 bp, Artificial Sequence
  synthetic oligonucleotide primer
aaacagacggccaacgtgcctcagac

SEQ ID NO: 13 - DNA, 26 bp, Artificial Sequence
  synthetic oligonucleotide primer
caccgtgcacgacctggtggccggcc

SEQ ID NO: 14 - DNA, 26 bp, Artificial Sequence
  synthetic oligonucleotide primer
aaacggccggccaccaggtcgtgcac

SEQ ID NO: 15 - DNA, 26 bp, Artificial Sequence
  synthetic oligonucleotide primer
caccgccagagacaacgccactggct

SEQ ID NO: 16 - DNA, 26 bp, Artificial Sequence
  synthetic oligonucleotide primer
aaacagccagtggcgttgtctctggc

SEQ ID NO: 17 - DNA, 21 bp, Artificial Sequence
  synthetic oligonucleotide primer
accgactggaagacacgtttg

SEQ ID NO: 18 - DNA, 19 bp, Artificial Sequence
  synthetic oligonucleotide primer
ccaggtcagcttcgcaagg

SEQ ID NO: 19 - DNA, 19 bp, Artificial Sequence
  synthetic oligonucleotide primer
tgaagcggctccctgtttt

SEQ ID NO: 20 - DNA, 20 bp, Artificial Sequence
  synthetic oligonucleotide primer
cgggtttctttcacaaggcg

SEQ ID NO: 21 - DNA, 22 bp, Artificial Sequence
  synthetic oligonucleotide primer
cagtaaggcaggctgtaaaaga

SEQ ID NO: 22 - DNA, 19 bp, Artificial Sequence
  synthetic oligonucleotide primer
tggagctggtagaccctcg

SEQ ID NO: 23 - DNA, 20 bp, Artificial Sequence
  synthetic oligonucleotide primer
ttgggcatagagaccccgtt

SEQ ID NO: 24 - DNA, 23 bp, Artificial Sequence
  synthetic oligonucleotide primer
gcacattgctcagttcatacacc

SEQ ID NO: 25 - DNA, 20 bp, Artificial Sequence
  synthetic oligonucleotide primer
tgtgggtctgggtaggaacc

SEQ ID NO: 26 - DNA, 21 bp, Artificial Sequence
  synthetic oligonucleotide primer
agctgtgtagcatagggcaag

SEQ ID NO: 27 - DNA, 21 bp, Artificial Sequence
  synthetic oligonucleotide primer
cagccagatgcaatcaatgcc

SEQ ID NO: 28 - DNA, 21 bp, Artificial Sequence
  synthetic oligonucleotide primer
tggaatcctgaacccacttct

SEQ ID NO: 29 - DNA, 22 bp, Artificial Sequence
  synthetic oligonucleotide primer
acgggtatgtggagatagagga

SEQ ID NO: 30 - DNA, 23 bp, Artificial Sequence
  synthetic oligonucleotide primer
ggacttttaggaagagccagact

SEQ ID NO: 31 - DNA, 20 bp, Artificial Sequence
  synthetic oligonucleotide primer
agaactgtgctgggaagacg

SEQ ID NO: 32 - DNA, 20 bp, Artificial Sequence
  synthetic oligonucleotide primer
gccatgccttcaaaatgcca

SEQ ID NO: 33 - DNA, 20 bp, Artificial Sequence
  synthetic oligonucleotide primer
cttcgaggcacaaggcacaa

SEQ ID NO: 34 - DNA, 20 bp, Artificial Sequence
  synthetic oligonucleotide primer
ttcactggcgagctcaggta

SEQ ID NO: 35 - DNA, 25 bp, Artificial Sequence
  synthetic oligonucleotide primer
ctcaatattagagtctcaaccccca

SEQ ID NO: 36 - DNA, 20 bp, Artificial Sequence
  synthetic oligonucleotide primer
gagaaggcaactggaccgaa

SEQ ID NO: 37 - DNA, 20 bp, Artificial Sequence
  synthetic oligonucleotide primer
ccaccggagcactccataag

SEQ ID NO: 38 - DNA, 20 bp, Artificial Sequence
  synthetic oligonucleotide primer
gatggttccttccggtggtt

SEQ ID NO: 39 - DNA, 20 bp, Artificial Sequence
  synthetic oligonucleotide primer
tgtgacggcagggaaatgta

SEQ ID NO: 40 - DNA, 23 bp, Artificial Sequence
  synthetic oligonucleotide primer
tctgctctaacacagagggaaac

SEQ ID NO: 41 - DNA, 21 bp, Artificial Sequence
  synthetic oligonucleotide primer
acggatttggtcgtattgggc

SEQ ID NO: 42 - DNA, 21 bp, Artificial Sequence
  synthetic oligonucleotide primer
ttgacggtgccatggaatttg

SEQ ID NO: 43 - DNA, 20 bp, Artificial Sequence
  synthetic oligonucleotide primer
cagggatacttggggttgct

SEQ ID NO: 44 - DNA, 21 bp, Artificial Sequence
  synthetic oligonucleotide primer
aggaagtccccaggactgtta

SEQ ID NO: 45 - DNA, 20 bp, Artificial Sequence
  synthetic oligonucleotide primer
atccccaacagacccgtttc

SEQ ID NO: 46 - DNA, 20 bp, Artificial Sequence
  synthetic oligonucleotide primer
gaacgcaggcttgcagaaaa

SEQ ID NO: 47 - DNA, 20 bp, Artificial Sequence
  synthetic oligonucleotide primer
gctacagtgtccctcgaacc

SEQ ID NO: 48 - DNA, 20 bp, Artificial Sequence
  synthetic oligonucleotide primer
ccggtgcatgtgtctcctta

SEQ ID NO: 49 - DNA, 21 bp, Artificial Sequence
  synthetic oligonucleotide primer
ccctgccctagctgcctaccg

SEQ ID NO: 50 - DNA, 20 bp, Artificial Sequence
  synthetic oligonucleotide primer
gcttcgcagggcctgaccat

SEQ ID NO: 51 - DNA, 20 bp, Artificial Sequence
  synthetic oligonucleotide primer
gccgtgggctgcattcctct

SEQ ID NO: 52 - DNA, 22 bp, Artificial Sequence
  synthetic oligonucleotide primer
gcggttcggtgccaaagacagg

SEQ ID NO: 53 - DNA, 20 bp, Artificial Sequence
  synthetic oligonucleotide primer
ctgccaaagccttcaatcgt

SEQ ID NO: 54 - DNA, 20 bp, Artificial Sequence
  synthetic oligonucleotide primer
ctagtagagggctggccttg

SEQ ID NO: 55 - DNA, 20 bp, Artificial Sequence
  synthetic oligonucleotide primer
gtgcagtgccagcctcgtcc

SEQ ID NO: 56 - DNA, 20 bp, Artificial Sequence
  synthetic oligonucleotide primer
gccactgcaaatggcagccc

SEQ ID NO: 57 - DNA, 22 bp, Artificial Sequence
  synthetic oligonucleotide primer
gtgatgctcaggtatccatcca

SEQ ID NO: 58 - DNA, 21 bp, Artificial Sequence
  synthetic oligonucleotide primer
cacagttctcaaagcacagcg

SEQ ID NO: 59 - DNA, 22 bp, Artificial Sequence
  synthetic oligonucleotide primer
tggtgactctggggataaagaa

SEQ ID NO: 60 - DNA, 23 bp, Artificial Sequence
  synthetic oligonucleotide primer
gggagtaactctgaatccgagac

SEQ ID NO: 61 - DNA, 20 bp, Artificial Sequence
  synthetic oligonucleotide primer
ggccctatcattcacgggag

SEQ ID NO: 62 - DNA, 22 bp, Artificial Sequence
  synthetic oligonucleotide primer
tctgaccggaattttcacctgt

SEQ ID NO: 63 - DNA, 21 bp, Artificial Sequence
  synthetic oligonucleotide primer
agttggggattcggttgttct

SEQ ID NO: 64 - DNA, 20 bp, Artificial Sequence
  synthetic oligonucleotide primer
cccctcattccttaccaccc

SEQ ID NO: 65 - DNA, 25 bp, Artificial Sequence
  synthetic oligonucleotide primer
atcccagcacagcaaatgctccaaa

SEQ ID NO: 66 - DNA, 20 bp, Artificial Sequence
  synthetic oligonucleotide primer
tggggtccgagggatgctgt

(19) United States
(12) Patent Application Publication
(10) Pub. No.: US 2018/0153993 A1
(43) Pub. Date: Jun. 7, 2018

(54) COMPOSITIONS AND METHODS FOR TREATMENT AND PREVENTION OF CARDIOVASCULAR DISEASES

(71) Applicant: The Chinese University of Hong Kong, Shatin (CN)

(72) Inventors: Yu Huang, Shatin (CN); Li Wang, Shatin (CN)

(21) Appl. No.: 15/789,853

(22) Filed: Oct. 20, 2017

Related U.S. Application Data
(60) Provisional application No. 62/431,094, filed on Dec. 7, 2016.

(51) Int. Cl. A61K 45/06; C07K 14/47; C07K 16/28; A61K 9/00; A61P 9/00

(52) U.S. Cl. CPC A61K 45/06; C07K 14/4703; A61P 9/00; C07K 16/2848; A61K 9/0053; C07K 14/4705

(57) ABSTRACT

The present invention provides novel methods for the prevention and treatment of cardiovascular diseases and inflammatory diseases by modulating the Hippo-YAP signaling pathway. Also provided are methods for identifying compounds that are capable of modulating the Hippo-YAP signaling pathway and are therefore useful for the prevention and treatment of cardiovascular diseases and inflammatory diseases.

### BACKGROUND OF THE INVENTION

This application claims priority to U.S. Provisional Patent Application No. 62/431,094, filed Dec. 7, 2016, the contents of which are herein incorporated in the entirety for all purposes.

### BACKGROUND OF THE INVENTION

Cardiovascular disease (CAD) is a term generally used to refer to medical conditions that affect the structures or function of the heart, such as coronary artery disease (narrowing of the arteries), heart attack, abnormal heart rhythms or arrhythmias, heart failure, heart valve disease, congenital heart disease, heart muscle disease (cardiomyopathy), pericardial disease, aorta disease and Marfan syndrome, as well as other vascular diseases (blood vessel diseases). One type of CAD is known as atherosclerosis, which involves the hardening of the arteries due to an excessive buildup of plaque around the artery wall. The disease disrupts the flow of blood around the body and can pose serious cardiovascular complications when arteries providing oxygen and nutrients to vital organs (e.g., heart) are impacted. Coronary artery disease, stroke, and peripheral artery disease involve atherosclerosis, which in turn may be caused by high blood pressure, smoking, diabetes, lack of exercise, obesity, high blood cholesterol, poor diet, and excessive alcohol consumption, among others. CAD is the leading cause of death globally and in North America.

Under normal physiological conditions, the inflammatory process works quickly to destroy and eliminate foreign and damaged cells, and to isolate the infected or injured tissues from the rest of the body. Inflammatory disorders arise when inflammation becomes uncontrolled and causes destruction of healthy tissue. Inflammatory disorders are diseases and conditions involving inflammation in an inappropriate manner, for example, many occur when the immune system mistakenly triggers inflammation in the absence of infection, such as inflammation of the joints in rheumatoid arthritis. In other examples, inflammatory disorders can result from a response to tissue injury or trauma but somehow affect the entire body. Inflammatory diseases include numerous specific diseases, such as Alzheimer's Disease, ankylosing spondylitis arthritis (osteoarthritis, rheumatoid arthritis (RA), psoriatic arthritis), asthma, atherosclerosis, Crohn's disease, colitis, dermatitis, diverticulitisfibromyalgia, hepatitis irritable bowel syndrome (IBS), systemic lupus erythematous (SLE), nephritis Parkinson's disease, ulcerative colitis, many of which can be painful, debilitating, and life-threatening.

Because of the prevalence of CAD and inflammatory diseases, especially considering their social economic impact as well as their grave implications on human life expectancy and quality of life, there exists an urgent need for developing new and more effective methods and therapeutic agents to prevent and treat these diseases. This invention fulfills this and other related needs.

### BRIEF SUMMARY OF THE INVENTION

The present inventors have identified the Hippo-YAP signaling pathway, especially effector YAP/TAZ, as a therapeutic target for the prevention and treatment of cardiovascular diseases (CAD) such as atherosclerosis, as well as various other related disorders and conditions such as inflammatory diseases. More specifically, the inventors show that, inhibition of YAP activity as well as activation of integrin β3 can suppress the development of CAD, inflammatory diseases, and various other associated disorders.

As such, in the first aspect, the present invention provides a method for treating or preventing a cardiovascular disease or an inflammatory disease in a subject. The method includes a step of administering to the subject a composition comprising an effective amount of an inhibitor of YAP or an activator of integrin β3. In some embodiments, the subject has been diagnosed with a cardiovascular disease or an inflammatory disease. In some embodiments, the subject is at risk of a cardiovascular disease or an inflammatory disease but has not been diagnosed with a cardiovascular disease or an inflammatory disease. In some embodiments, the composition is a medicament, for example, a medicine formulated to be administered by way of injection (e.g., intravenous, intramuscular, or subcutaneous) or oral ingestion. In some embodiments, the composition is a dietary supplement administered by oral ingestion, especially in the case of administration to a subject at risk but not yet diagnosed with a CAD or an inflammatory disease.

In a second aspect, the present invention provides a method for identifying a modulator of integrin-YAP/TAZ signaling pathway. The method includes these steps: (a) placing an endothelial cell under unidirectional shear stress; (b) contacting the cell with a candidate compound and determining YAP phosphorylation level at Ser127; and (c) comparing the YAP phosphorylation level at Ser127 obtained in step (b) with YAP phosphorylation level at Ser127 in a control endothelial cell under unidirectional shear stress but not contacted with the candidate compound; and (d) determining the candidate compound as an inhibitor of YAP or activator of integrin β3 when the YAP phosphorylation level at Ser127 obtained in step (b) is greater than the YAP phosphorylation level at Ser127 in the control endothelial cell, and determining the candidate compound as an activator of YAP or inhibitor of integrin β3 when the YAP phosphorylation level at Ser127 obtained in step (b) is less than the YAP phosphorylation level at Ser127 in the control endothelial cell.

In a third aspect, the present invention provides another method for identifying a modulator of integrin-YAP/TAZ signaling pathway. The method includes these steps: (a) placing an endothelial cell under disturbed flow; (b) contacting the cell with a candidate compound and determining YAP phosphorylation level at Ser127; and (c) comparing the YAP phosphorylation level at Ser127 obtained in step (b) with YAP phosphorylation level at Ser127 in a control endothelial cell under disturbed flow but not contacted with the candidate compound; and (d) determining the candidate compound as an inhibitor of YAP or activator of integrin β3 when the YAP phosphorylation level at Ser127 obtained in step (b) is greater than the YAP phosphorylation level at Ser127 in the control endothelial cell, and determining the candidate compound as an activator of YAP or inhibitor of integrin β3 when the YAP phosphorylation level at Ser127 obtained in step (b) is less than the YAP phosphorylation level at Ser127 in the control endothelial cell.

In a fourth aspect, the present invention provides yet another method for identifying a modulator of integrin-YAP/TAZ signaling pathway. The method comprises these steps: (a) placing an endothelial cell under unidirectional shear stress; (b) contacting the cell with a candidate compound and determining integrin β3-Gα13 association level; (c) comparing the integrin β3-Gα13 association level obtained in step (b) with integrin β3-Gα13 association level in a control endothelial cell under unidirectional shear stress but not contacted with the candidate compound; and (d) determining the candidate compound as an inhibitor of YAP or activator of integrin β3 when the integrin β3-Gα13 association level obtained in step (b) is greater than the integrin β3-Gα13 association level in the control endothelial cell, and determining the candidate compound as an activator of YAP or inhibitor of integrin β3 when the integrin β3-Gα13 association level obtained in step (b) is less than the integrin β3-Gα13 association level in the control endothelial cell.

In a fifth aspect, the present invention provides still another method for identifying a modulator of integrin-YAP/TAZ signaling pathway. The method comprises these steps: (a) placing an endothelial cell under disturbed flow; (b) contacting the cell with a candidate compound and determining integrin β3-Gα13 association level; (c) comparing the integrin β3-Gα13 association level obtained in step (b) with integrin β3-Gα13 association level in a control endothelial cell under disturbed flow but not contacted with the candidate compound; and (d) determining the candidate compound as an inhibitor of YAP or activator of integrin β3 when the integrin β3-Gα13 association level obtained in step (b) is greater than the integrin β3-Gα13 association level in the control endothelial cell, and determining the candidate compound as an activator of YAP or inhibitor of integrin β3 when the integrin β3-Gα13. association level obtained in step (b) is less than the integrin β3-Gα13 association level in the control endothelial cell.

In some embodiments of any one of the screening methods described above and herein, the endothelial cell used in the screening assay is a human umbilical vein endothelial cell (HUVEC) or human aortic endothelial cell (HAEC).

In a sixth aspect, the present invention provides a kit for treating or preventing a cardiovascular disease or an inflammatory disease in a subject. The kit typically includes (1) a composition comprising an effective amount of an activator of YAP or inhibitor of integrin β3, such as one modulator of integrin-YAP/TAZ signaling pathway identified by the screening methods of this invention; and optionally (2) another agent effective for treating or preventing a cardiovascular disease or an inflammatory disease. The two agents may be kept in the same or separate containers. In some embodiments, the kit further comprises an instruction manual to provide information for the user in the administration of a modulator of integrin-YAP/TAZ signaling pathway.

### BRIEF DESCRIPTION OF THE DRAWINGS

FIG. 1A-1I: Haemodynamics regulates YAP phosphorylation, subcellular localization, downstream gene expression and reporter gene activity in ECs. FIG. 1A, Immunoblotting showing YAP expression is higher in mouse aorta with endothelium (+endo) than that without endothelium (−endo). FIG. 1B, USS promotes, while disturbed flow inhibits, YAP phosphorylation. FIG. 1C, USS promotes YAP nuclear exportation in HUVECs. YAP was visualized by immunostaining (green); nuclei were counterstained with propidium iodide (PI, red). FIGS. 1D and 1E, USS suppresses while disturbed flow increases (FIG. 1D) 8*GTIIC-luc reporter gene activity and (FIG. 1E) expression of YAP/TAZ target genes CTGF and CYR61 (n=3; compared with static (STA), *P<0.05 by two-tailed unpaired t-test). FIG. 1F, Immunoblotting showing YAP phosphorylation level is higher in thoracic aorta (TA, straight) compared with aortic arch (AA, curved) from C57BL/6J mice. FIG. 1G, En face staining of YAP in mouse aorta showing increased YAP nuclear localization in inner curvature of the aortic arch compared with outer curvature and thoracic aorta (n(TA)=6, n(AA inner)=3, n(AA outer)=3). FIGS. 1H and 1I, (FIG. 1I) Immunostaining of pYAP in (FIG. 1H) rat abdominal aorta with surgical stenosis, showing increased pYAP in the clipped region and decreased pYAP in the downstream region (n=3). Representative images of three separate experiments are shown.

FIGS. 2A-2L: Integrin inhibits YAP/TAZ activity through Gα₁₃-mediated RhoA inhibition. FIG. 2A, Cytoplasmic-domain-deleted integrin (β3Δcyto) reverses USS-induced YAP phosphorylation in HUVECs. FIG. 2B, RGD-containing peptide GRGDSP (100 μg ml⁻¹) induces YAP phosphorylation. FIG. 2C, Knockdown of Gα₁₃ or integrin β₃ attenuates MnCl₂-induced (0.5 mM for 5 min) YAP phosphorylation. FIG. 2D, Integrin β₃ Pro32Pro33 mutation induces YAP phosphorylation. FIGS. 2E and 2F, CA-RhoA suppresses YAP phosphorylation induced by (FIG. 2E) USS or (FIG. 2F) 0.5 mM MnCl₂. FIGS. 2G and 2H, Gα₁₃ knockdown reverses (FIG. 2G) USS-induced YAP phosphorylation and (FIG. 2H) MnCl₂-induced GTP-RhoA suppression. FIG. 2I, Gα₁₃ inhibiting peptide SRI overexpression reverses USS-induced YAP phosphorylation. j, The expression of integrin β₃, Gα₁₃, pYAP, YAP and TAZ in atherosclerotic aorta of ApoE^{-/-} mice on Western diet (WD) or normal diet (ND) for 3 months. FIGS. 2K and 2L, pYAP level in atherosclerotic lesions of (FIG. 2K) ApoE^{-/-} mice (n=5) and (FIG. 2L) human (n=5). pYAP (pYAP(red for FIG. 2K and green for FIG. 2I), vWF (red), VCAM1 (red) and α-SMA (green) were visualized by immunostaining; nuclei were counterstained with DAPI (blue). The representative images of at least three separate experiments are shown.

FIGS. 3A-3H: YAP/TAZ activation induces adhesion molecule expression through increasing JNK activity. FIG. 3A, KEGG enrichment pathway analysis and (FIG. 3B) Gene Ontology (GO) enrichment analysis for mRNA profile in HUVECs transfected with CA-YAP/TAZ c, JNK inhibitor SP600125 suppresses CA-YAP/TAZ-induced inflammatory gene expression in HUVECs (n=3; *P<0.05 by two-tailed unpaired t-test). FIG. 3D, CA-YAP/TAZ promotes monocyte attachment to HUVECs. FIGS. 3E and 3F, YAP/TAZ knockdown reduces expression of JNK target genes (IL6 and IL8) and (FIG. 3F) AP-1 reporter gene activity induced by PMA (n=3; *P<0.05 by two-tailed unpaired t-test). FIGS. 3G and 3H, EC-specific YAP overexpression promotes (FIG. 3G) atherosclerotic plaque formation, visualized by Oil Red O staining and (FIG. 3H) JNK activation, detected by immunostaining of pJNK (red) (n=5, representative result is shown).

FIGS. 4A-4G: Suppression of YAP/TAZ activity retards atherogenesis. FIGS. 4A and 4B, AAV-mediated CRISPR/Cas9 system specifically knocks down YAP level in endothelium of ApoE^{-/-} mice. Illustration (FIG. 4A, left) showing carotid partial ligation surgery in ApoE^{-/-} mice. YAP knockdown was confirmed by (FIG. 4A, right) immunostaining (YAP (green), Vcaml(red), nuclei(blue)) (n=5, representative result is shown) and (FIG. 4B) immunoblotting of YAP in aorta. FIG. 4C, EC-specific YAP knockdown reduces plaque formation in ApoE^{-/-} mice receiving carotid partial ligation (arrow) surgery. FIG. 4D, Oral administration of MnCl₂ decreases atherosclerotic plaque formation visualized by Oil Red O staining. FIG. 4E, YAP/TAZ reporter gene activity assay of anti-atherosclerotic agents showing statins produce the strongest inhibitory effect on YAP/TAZ activity (n=3; *P<0.05 by two-tailed unpaired t-test). FIG. 4F, Simvastatin suppresses expression of YAP/TAZ target genes while failing to reverse CA-YAP/TAZ-induced expression of pro-inflammatory genes (n=3; *P<0.05 by two-tailed unpaired t-test). FIG. 4G, Illustration of the haemodynamics-regulated YAP/TAZ signalling in ECs.

FIGS. 5A-5H: USS and disturbed flow oppositely regulate YAP/TAZ activity. FIG. 5A, Immunoblotting showing USS induces YAP phosphorylation in human aortic ECs. FIG. 5B, Summarized data for USS-induced YAP nuclear exportation. FIG. 5C, TAZ is decreased in nuclear fractions and increased in cytoplasmic fractions in HUVECs exposed to USS for 6 h. TAZ expression was detected by immunoblotting after cell fractionation. FIG. 5D, Disturbed flow suppresses YAP phosphorylation in human aortic ECs. e, Immunoblotting showing disturbed flow increases CTGF expression in HUVECs. All immunoblotting experiments were repeated three times and the representative results are shown. FIGS. 5F and 5G, YAP/TAZ knockdown attenuates gene expression of disturbed-flow-induced (FIG. 5F) CTGF and (FIG. 5G) CYR61 (n=3; *P<0.05 by two-tailed unpaired t-test). FIG. 5H, Summarized data for en face staining of relative nuclear YAP level in mouse aorta.

FIGS. 6A-6I: USS inhibits YAP/TAZ through integrin-Gα₁₃-RhoA pathway. FIG. 6A, MnCl₂ (0.5 mM) promotes YAP phosphorylation shown by immunoblotting. FIG. 6B, MnCl₂ reduces nuclear YAP/TAZ levels in HUVECs. FIG. 6C, Gα₁₃ inhibiting peptide mSRI reverses MnCl₂-induced YAP/TAZ reporter (8×GTIIC-luc) gene activity (n=3; *P<0.05 by two-tailed unpaired t-test). FIG. 6D, RGD containing peptide GRGDSP downregulates YAP/TAZ downstream target gene expression (n=3; *P<0.05 by two-tailed unpaired t-test). FIGS. 6E and 6F, Pro32pro33 mutation in integrin β₃ inhibits YAP/TAZ transactivation in HUVECs, as verified by suppressed (FIG. 6E) expression of YAP/TAZ target genes and (FIG. 6F) YAP/TAZ reporter gene activity (n=3; *P<0.05 by two-tailed unpaired t-test). FIG. 6G, Gα₁₃ or integrin β₃ knockdown reverses MnCl₂-induced YAP/TAZ nuclear exportation in HUVECs. FIG. 6H, Gα₁₃ knockdown reverses RGD containing peptide-mediated CTGF and CYR61 suppression in HUVECs (n=3; *P<0.05 by two-tailed unpaired t-test). FIG. 6I, Gα₁₃ inhibiting peptide mSRI and mP6 reverse MnCl₂-induced (5 min) pYAP but not total YAP expression in HUVECs. The experiments were repeated at least three times and the representative results are shown.

FIGS. 7A-7H: YAP/TAZ activation increases JNK activity. FIG. 7A, Heat map for mRNA sequencing results showing CA-YAP/TAZ promotes expression of pro-inflammatory genes. FIG. 7B, CA-YAP/TAZ increases the promoter activity of adhesion molecules in HUVECs. FIG. 7C, Summarized data for CA-YAP/TAZ overexpression increases monocyte attachment to HUVECs. FIGS. 7D and 7E, Immunoblotting showing JNK phosphorylation in HUVECs exposed to (FIG. 7D) USS or (FIG. 7E) disturbed flow for different durations. Experiments were repeated three times and the representative results are shown. FIG. 7F, YAP/TAZ knockdown suppresses basal and PMA-induced JNK phosphorylation in HUVECs. FIG. 7G, Overexpression of dominant negative YAP (YAP S94A) inhibits PMA-induced AP-1 reporter gene activity. FIG. 7H, CA-YAP/TAZ increases AP-1 reporter gene activity in HUVECs (n=4; *P<0.05 by two-tailed unpaired t-test), and PMA was used as positive control for monitoring AP-1 activity.

FIGS. 8A-8F: EC-specific overexpression of YAP accelerates plaque formation. FIG. 8A, The generation of Cre-mediated EC-specific YAP overexpression transgenic mice. FIG. 8B, En face staining showing increased YAP expression in endothelial cells of the Tie2^{Cre/+}; YAP-COE^{g/+}; ApoE^{-/-} (n=10) FIG. 8C, Summarized data for EC-specific YAP overexpression-increased JNK phosphorylation. FIG. 8D, EC-specific YAP overexpression increases macrophage content in the atherosclerotic plaques from aortic root. FIGS. 8E and 8F, EC-specific YAP overexpression does not affect serum levels of (FIG. 8E) cholesterol or (FIG. 8F) triglycerides.

FIGS. 9A-9I: Inhibiting TAZ activity by shRNA or MnCl₂ administration delays atherogenesis and is independent of lipid metabolism, while activating YAP/TAZ by AAV-mediated CA-YAP/TAZ overexpression accelerates atherosclerotic plaque formation. FIG. 9A, Immunoblotting showing adenovirus-mediated TAZ shRNA suppressed TAZ expression level. FIG. 9B, TAZ knockdown delayed Western-diet-induced plaque formation in ApoE^{-/-} mice. FIG. 9C, TAZ knockdown suppressed plaque formation in ApoE^{-/-} mice is not due to change in lipid profile. FIG. 9D, Immunoblotting showing increased YAP expression in mice injected with AAV expressing CA-YAP/TAZ. FIG. 9E and 9F, (FIG. 9E) Oil Red O staining and (FIG. 9F) summarized data for CA-YAP/TAZ-induced exacerbation of plaque formation. FIG. 9G. AAV-mediated CA-YAP/TAZ overexpression does not affect lipid profile in ApoE^{-/-} mice. FIGS. 9H and 9I, Oral administration of MnCl₂ does not affect (FIG. 9H) lipid profile or (FIG. 9I) SOD activity in liver. Data are expressed as mean±s.e.m., n=5−6; *P<0.05 by two-tailed unpaired t-test.

FIGS. 10A-10I: Summary of western blotting data. FIG. 10A, Endothelium removal reduces YAP level in mouse aorta. FIG. 10B, USS increases YAP phosphorylation. FIG. 10C, Disturbed flow reduces YAP phosphorylation. FIG. 10D, Thoracic aorta expresses higher levels of pYAP than aortic arch. FIG. 10E, Overexpression loss-of-function mutation of integrin β₃ (β₃Δcyto) suppresses USS induced pYAP. FIG. 10F, RGD containing peptide GRGDSP induces pYAP. FIG. 10G, Gα₁₃ or integrin β₃ knockdown reverses MnCl₂-induced pYAP. FIG. 10H, Integrin gain-of-function mutation Pro32Pro33 increases pYAP. FIG. 10I, Constitutively activated RhoA (CA-RhoA) reverses USS-induced pYAP. Data: n=6 for FIG. 10A and n=3 for other figures; *P<0.05 by two-tailed unpaired t-test.

FIGS. 11A-11I: Summary of western blotting data. FIG. 11A, CA-RhoA reverses MnCl₂-induced pYAP. FIG. 11B, Gα₁₃ knockdown reverses USS-induced pYAP. FIG. 11C, Gα₁₃ inhibitor SRI reverses USS-induced pYAP. FIGS. 11D-11H, Immunoblotting detection of (FIG. 11D) pYAP, (FIG. 11E) YAP, (FIG. 11F) TAZ, (FIG. 11G) Gα₁₃ and (FIG. 11H) integrin β₃ levels. FIG. 11I, YAP knockdown by the CRISPR-Cas9 in vivo genome editing system. Data: n=3 for FIGS. 11A-11C, n=5 for FIGS. 11D-11I; *P<0.05 by two-tailed unpaired t-test.

### DEFINITIONS

The term “treat” or “treating,” as used in this application, describes to an act that leads to the elimination, reduction, alleviation, reversal, or prevention or delay of onset or recurrence of any symptom of a relevant condition. In other words, “treating” a condition encompasses both therapeutic and prophylactic intervention against the condition.

The term “effective amount” as used herein refers to an amount of a given substance that is sufficient in quantity to produce a desired effect. For example, an effective amount of an inhibitor of YAP or an activator of integrin β3 is the amount of said inhibitor or activator to achieve its intended biological activity, such that the symptoms of a cardiovascular disease or an inflammatory disease are reduced, reversed, eliminated, prevented, or delayed of the onset in a patient who has been given the inhibitor or activator for therapeutic purposes. An amount adequate to accomplish this is defined as the “therapeutically effective dose.” The dosing range varies with the nature of the therapeutic agent being administered and other factors such as the route of administration and the severity of a patient's condition.

The term “subject” or “subject in need of treatment,” as used herein, includes individuals who seek medical attention due to risk of, or actual suffering from, a relevant disease or condition, e.g., a cardiovascular disease or an inflammatory disease. Subjects also include individuals currently undergoing therapy that seek manipulation of the therapeutic regimen.

Subjects or individuals in need of treatment include those that demonstrate symptoms of the relevant disease or are at risk of suffering from the disease or its symptoms. For example, a subject in need of treatment includes individuals with a genetic predisposition or family history for cardiovascular or inflammatory diseases, those that have suffered relevant symptoms in the past, those that have been exposed to a triggering substance or event, as well as those suffering from chronic or acute symptoms of the condition. A “subject in need of treatment” may be at any age of life.

“Inhibitors,” “activators,” and “modulators” of YAP or integrin β3 are used to refer to inhibitory, activating, or modulating molecules, respectively, identified using in vitro and in vivo assays for YAP phosphorylation or for integrin β3-Gα13 protein binding/association, especially as observed in endothelial cells under disturbed flow or unidirectional shear stress. The term “modulator” includes inhibitors and activators. Inhibitors are agents that, e.g., partially or totally block the activity of a target protein, such as YAP protein or integrin β3 (manifested in increased YAP phosphorylation at Ser127 or decreased association between integrin β3 and Gα13, respectively). In some cases, the inhibitor directly or indirectly binds to the protein, such as a neutralizing antibody. Inhibitors, as used herein, are synonymous with inactivators and antagonists. Activators are agents that, e.g., stimulate, increase, facilitate, enhance activation, sensitize or up regulate the activity of the target protein, such as YAP protein or integrin β3 (manifested in decreased YAP phosphorylation at Ser127 or increased association between integrin β3 and Gα13, respectively). Modulators include target protein ligands or binding partners, including modifications of naturally-occurring ligands and synthetically-designed ligands, antibodies and antibody fragments, antagonists, agonists, small molecules including carbohydrate-containing molecules, siRNAs, RNA aptamers, and the like.

As used herein, the terms “unidirectional shear stress (USS)” and “disturbed flow” describe fluid flow patterns and their effect on surrounding surface, e.g., blood flow patterns in the circulatory system in relation to cells, especially endothelial cells lining the inner surface of the blood vessels. In this context, “unidirectional shear stress” refers to, when a fluid (e.g., blood) flows in one direction along a flow path (e.g., a blood vessel such as an artery), the force on the parallel inner surface (e.g., endothelial cell surface) of the flow path. In contrast, “disturbed flow” refers to a more complex fluid flow pattern without one definitive direction due to irregularities or certain geometries present in a flow path such as a branching point of a blood vessel or a partial obstacle (e.g., plaque buildup) in the blood vessel, resulting in the fluid flow having multiple directions. This more complex flow pattern leads to forces on the surface of the flow path (e.g., endothelial cell surface) being different from the forces on the surface due to unidirectional shear stress.

### DETAILED DESCRIPTION OF THE INVENTION

### Introduction

The present inventors discovered for the first time that the Hippo-YAP signaling pathway is involved in the pathogenesis of cardiovascular diseases and inflammatory diseases. The present invention thus relates to preventing or reducing atherosclerosis and other cardiovascular and metabolic diseases by using inhibitors of the Hippo-YAP pathway. The Hippo pathway is a newly identified signaling pathway which plays a role in the control of organ size and development. Recent study by the present inventors provides novel evidence demonstrating that the Hippo pathway effector YAP/TAZ are activated in endothelial cells from human and mouse with cardiovascular and metabolic dysfunction, such as atherosclerosis.

Cardiovascular disease (CAD) is a class of heart and blood vessels diseases. Higher level of adhesion molecules expression is closely associated with the initiation and development of atherosclerosis. The inventors have found a large amount of adhesion molecules cells are controlled by YAP/TAZ signaling in endothelial cells. In ApoE−/− mice, specific over-expression of endothelial YAP accelerated formation of atherosclerotic plaques. By contrast, inhibition of YAP/TAZ via virus-mediated shRNA or CRISPR/Cas9 reversed metabolic disorder in mice of experimentally induced atherosclerosis. Suppression of YAP/TAZ activity by orally administration of MnCl₂ ameliorated plaques formation in atherosclerotic ApoE−/− mice, indicating that pharmacologically intervention of the Hippo pathway is very effective to regress atherosclerosis.

Based on this discovery, an anti-CAD drug screening platform has been established, and the inventors have constructed an adenovirus-mediated reporter gene system for semi-automatic, high throughput screening platform. An initial trial for several anti-atherosclerotic agents identified that some of these drugs exert an anti-atherosclerotic effect. Statins, for example, exhibited the strongest YAP/TAZ inhibitory effect. Since statins are the first-line therapy for cardiovascular diseases, these results indicate that this novel drug screening platform may lead to identification of effective compounds for the treatment of CAD as well as other diseases and conditions such as inflammatory diseases, for example, atherosclerosis, hypertension, metabolic diseases, such as obesity, diabetes, obesity/diabetes-induced vascular dysfunction, in patients.

The present invention of targeting the Hippo pathway provides a novel strategy against cardiovascular diseases and other related diseases and disorders. Currently, cholesterol-lowering therapy is most commonly used to treat patients with dyslipidemia and atherosclerosis. However, patients suffer from some side effects during drug treatment. Moreover, a substantial number of people with normal cholesterol levels (<200 mg/dL) develop coronary artery disease. By contract, a significant number of individuals with elevated cholesterol (225-300 mg/dL) do not have coronary artery disease. It is, therefore, necessary to identify alternative drug targets.

Adenovirus-mediated reporter system and immortalized human aortic endothelial cells (HAECs) are used for drug screening. The present inventors have found that increased YAP/TAZ activity in endothelial cells is closely associated with the development of atherosclerosis and plaques formation. Endothelial cells express specific receptors that determine their unique response to different drug treatment. Therefore, it is necessary to use endothelial cells to study the YAP/TAZ inhibitory effect of lead drugs. Since primary endothelial cells from different donors contain distinct genetic background, which might cause significant variation between different batches of experiments, the immortalized human aortic endothelial cells are used. However, endothelial cells are difficult to be transformed, which could delay research progress and increase cost. To overcome this problem, the inventors have generated adenoviral expressing YAP/TAZ luciferase reporter and renilla internal control. The entire screening process can be completed in two days.

### II. Identification of Modulators of the Hippo-YAP Signaling Pathway

By illustrating the correlation of YAP/TAZ signaling and atherogenesis and inflammation, the present invention provides a means for treating patients suffering from cardiovascular diseases or inflammatory diseases or for reducing the risk of later developing such diseases: by way of inhibiting YAP/TAZ activity and/or increasing integrin β3-Gα13 association and biological activity. As used herein, treatment of the pertinent diseases encompasses reducing, reversing, lessening, or eliminating one or more of the symptoms of the diseases, as well as preventing or delaying the onset of one or more of the relevant symptoms.

In a closely related aspect, the present invention provides a method for identifying modulators of the Hippo-YAP signaling pathway, for example, inhibitors of YAP and activators of integrin β3, as these compounds are useful for modulating YAP signaling and are therefore useful for treating cardiovascular diseases, inflammatory diseases, and other related conditions and disorders or for reducing the risk of developing such diseases. These modulators may be of any chemical nature, small molecules or macromolecules.

In general, a candidate compound is first tested in an in vitro assay, for example, a cell-based assay system, for any potential positive or negative affect on various molecules in the Hippo-YAP signaling pathway. An endothelial cell is typically used in such assay systems for its natural expression of the molecules in the Hippo-YAP pathway and for its natural response to stimuli such as unidirectional shear stress (USS) or disturbed flow. Virtually any mammalian endothelial cells can be used, with human umbilical vein endothelial cell (HUVEC) or human aortic endothelial cell (HAEC) being two examples. Typically, endothelial cells are first cultured on a slide (e.g., a glass or plastic slide), the slide is then placed in a flow chamber and subject to an appropriate flow pattern (such as disturbed flow or unidirectional shear stress) for an appropriate time duration (e.g., at least 5, 10, or 15 minutes, or from 10 minutes to 20, 30, or 60 minutes, or 1-2 hours, or 2-3 hours, or 3-6 hours). Instruments for performing cell-based assays under different flow patterns are available via commercial suppliers such as

Disturbed flow leads to activation of YAP/TAZ, as shown in (1) decreased integrin β3-Gα13 association, and (2) decreased phosphorylation of YAP at Ser127; whereas unidirectional shear stress leads to suppression of YAP/TAZ, as shown in (1) increased integrin β3-Gα13 association, and (2) increased phosphorylation of YAP at Ser127. These changes can serve as indicators of a test compound's potential as either an inhibitor of YAP or an activator of integrin β3: if the presence of the test compound leads to increased YAP phosphorylation in comparison with the YAP phosphorylation level in the absence of the compound, then the compound is a potential inhibitor of YAP. Similarly, if the presence of a test compound leads to increased integrin β3 association with Gα13 in comparison to the integrin β3-Gα13 association level in the absence of the compound, the candidate compound is possibly an activator of integrin β3.

Once the cell-based screening is performed and provides indications of which compounds are likely modulators of the Hippo-YAP signaling pathway, additional testing (e.g., in vivo or animal-based testing) may be performed on these compounds to further confirm their capability of modulating YAP signaling. Once confirmed, the inhibitors/activators then can be employed in various therapeutic and prophylactic applications.

As stated above, a modulator of the integrin-YAP signaling pathway (such as an inhibitor of YAP or an activator of integrin β3) can have diverse chemical and structural features. For instance, an inhibitor can be a non-functional YAP protein mutant (e.g., a dominant negative mutant), an antibody to the YAP protein that interferes with YAP protein activity (e.g., a neutralizing antibody), or any small molecule or macromolecule that simply hinders the interaction between YAP protein and its cofactors or other binding partners. Essentially any chemical compound can be tested as a potential inhibitor of YAP protein activity. Most preferred are generally compounds that can be dissolved in aqueous or organic (especially DMSO-based) solutions. Inhibitors can be identified by screening a combinatorial library containing a large number of potentially effective compounds. Such combinatorial chemical libraries can be screened in one or more assays, as described herein, to identify those library members (particular chemical species or subclasses) that display a desired characteristic activity. The compounds thus identified can serve as conventional “lead compounds” or can themselves be used as potential or actual therapeutics. Similarly, an activator of integrin β3 may be a macromolecule such as a mutant integrin protein that is constitutively activated, or may be a small molecule that enhances the interaction between integrin β3 and Gα13. The activators may be identified by way of screening combinatorial chemical libraries in one or more assays, as described herein or known in the pertinent research field.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. I Pept. Prot. Res. 37:487-493 (1991) and Houghton et al., Nature 354:84-88 (1991)) and carbohydrate libraries (see, e.g., Liang et al., Science, 274:1520-1522 (1996) and U.S. Pat. No. 5,593,853). Other chemistries for generating chemical diversity libraries can also be used. Such chemistries include, but are not limited to: peptoids (PCT Publication No. WO 91/19735), encoded peptides (PCT Publication WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with β-D-glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see, Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; and benzodiazepines, U.S. Pat. No. 5,288,514).

### III. Pharmaceutical Compositions

### 1. Formulations

Modulators of the integrin-Gα13-YAP pathway of this invention are useful in the manufacture of a pharmaceutical composition or a medicament. A pharmaceutical composition or medicament can be administered to a subject for the treatment of a pertinent disease or for reducing the risk of at a later time developing such a disease, e.g., a cardiovascular disease or inflammatory disease.

Compounds used in the present invention, e.g., an inhibitor of YAP or an activator of integrin β3, are useful in the manufacture of a pharmaceutical composition or a medicament comprising an effective amount thereof in conjunction or mixture with excipients or carriers suitable for application.

An exemplary pharmaceutical composition for inhibiting YAP/activating integrin β3 comprises (i) an inhibitor of YAP or an activator of integrin β3, and (ii) a pharmaceutically acceptable excipient or carrier. The terms pharmaceutically-acceptable and physiologically-acceptable are used synonymously herein. The inhibitor or activator may be provided in a therapeutically effective dose for use in a method for treatment as described herein.

An inhibitor of YAP or an activator of integrin β3 can be administered via liposomes, which serve to target the modulator to a particular tissue, as well as increase the half-life of the composition. Liposomes include emulsions, foams, micelles, insoluble monolayers, liquid crystals, phospholipid dispersions, lamellar layers and the like. In these preparations the inhibitor or activator to be delivered is incorporated as part of a liposome, alone or in conjunction with a molecule which binds to, e.g., a receptor prevalent among the targeted cells (e.g., endothelial cells), or with other therapeutic compositions. Thus, liposomes filled with a desired inhibitor/activator of the invention can be directed to the site of treatment, where the liposomes then deliver the selected inhibitor/activator compositions. Liposomes for use in the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al. (1980) Ann. Rev. Biophys. Bioeng. 9: 467, U.S. Pat. Nos. 4,235,871, 4,501,728 and 4,837,028.

Pharmaceutical compositions or medicaments for use in the present invention can be formulated by standard techniques using one or more physiologically acceptable carriers or excipients. Suitable pharmaceutical carriers are described herein and in “Remington's Pharmaceutical Sciences” by E. W. Martin. Inhibitors or activator of the present invention and their physiologically acceptable salts and solvates can be formulated for administration by any suitable route, including via inhalation, topically, nasally, orally, parenterally, or rectally.

Typical formulations for topical administration include creams, ointments, sprays, lotions, and patches. The pharmaceutical composition can, however, be formulated for any type of administration, e.g., intradermal, subdermal, intravenous, intramuscular, intranasal, intracerebral, intratracheal, intraarterial, intraperitoneal, intravesical, intrapleural, intracoronary or intratumoral injection, with a syringe or other devices. Formulation for administration by inhalation (e.g., aerosol), or for oral, rectal, or vaginal administration is also contemplated.

### 2. Routes of Administration

Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

Suitable formulations for transdermal application include an effective amount of a inhibitor or activator of the present invention with carrier. Preferred carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used.

For oral administration, a pharmaceutical composition or a medicament can take the form of, for example, a tablet or a capsule prepared by conventional means with a pharmaceutically acceptable excipient. Preferred are tablets and gelatin capsules comprising the active ingredient, i.e., an inhibitor of YAP or an activator of integrin β3, together with (a) diluents or fillers, e.g., lactose, dextrose, sucrose, mannitol, sorbitol, cellulose (e.g., ethyl cellulose, microcrystalline cellulose), glycine, pectin, polyacrylates and/or calcium hydrogen phosphate, calcium sulfate, (b) lubricants, e.g., silica, talcum, stearic acid, its magnesium or calcium salt, metallic stearates, colloidal silicon dioxide, hydrogenated vegetable oil, corn starch, sodium benzoate, sodium acetate and/or polyethyleneglycol; for tablets also (c) binders, e.g., magnesium aluminum silicate, starch paste, gelatin, tragacanth, methylcellulose, sodium carboxymethylcellulose, polyvinylpyrrolidone and/or hydroxypropyl methylcellulose; if desired (d) disintegrants, e.g., starches (e.g., potato starch or sodium starch), glycolate, agar, alginic acid or its sodium salt, or effervescent mixtures; (e) wetting agents, e.g., sodium lauryl sulphate, and/or (f) absorbents, colorants, flavors and sweeteners.

Tablets may be either film coated or enteric coated according to methods known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound.

Inhibitors or activators of the present invention can be formulated for parenteral administration by injection, for example by bolus injection or continuous infusion. Formulations for injection can be presented in unit dosage form, for example, in ampoules or in multi-dose containers, with an added preservative. Injectable compositions are preferably aqueous isotonic solutions or suspensions, and suppositories are preferably prepared from fatty emulsions or suspensions. The compositions may be sterilized and/or contain adjuvants, such as preserving, stabilizing, wetting or emulsifying agents, solution promoters, salts for regulating the osmotic pressure and/or buffers. Alternatively, the active ingredient can be in powder form for constitution with a suitable vehicle, for example, sterile pyrogen-free water, before use. In addition, they may also contain other therapeutically valuable substances. The compositions are prepared according to conventional mixing, granulating or coating methods, respectively, and contain about 0.1 to 75%, preferably about 1 to 50%, of the active ingredient.

For administration by inhalation, the active ingredient, e.g., an inhibitor of YAP or an activator of integrin β3, may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound and a suitable powder base, for example, lactose or starch.

The inhibitors/activators can also be formulated in rectal compositions, for example, suppositories or retention enemas, for example, containing conventional suppository bases, for example, cocoa butter or other glycerides.

Furthermore, the active ingredient can be formulated as a depot preparation. Such long-acting formulations can be administered by implantation (for example, subcutaneously or intramuscularly) or by intramuscular injection. Thus, for example, the active ingredient can be formulated with suitable polymeric or hydrophobic materials (for example as an emulsion in an acceptable oil) or ion exchange resins, or as sparingly soluble derivatives, for example, as a sparingly soluble salt.

A pharmaceutical composition or medicament of the present invention comprises (i) an effective amount of an inhibitor of YAP or an activator of integrin β3, and (ii) another therapeutic agent. When used with a compound of the present invention, such therapeutic agent may be used individually, sequentially, or in combination with one or more other such therapeutic agents (e.g., a first therapeutic agent, a second therapeutic agent, and a compound of the present invention). Administration may be by the same or different route of administration or together in the same pharmaceutical formulation.

### 3. Dosage

Pharmaceutical compositions or medicaments can be administered to a subject at a therapeutically effective dose to prevent, treat, or control gastric cancer as described herein. The pharmaceutical composition or medicament is administered to a subject in an amount sufficient to elicit an effective therapeutic response in the subject.

The dosage of active agents administered is dependent on the subject's body weight, age, individual condition, surface area or volume of the area to be treated and on the form of administration. The size of the dose also will be determined by the existence, nature, and extent of any adverse effects that accompany the administration of a particular compound in a particular subject. For example, each type of YAP inhibitor or integrin β3 activator will likely have a unique dosage. A unit dosage for oral administration to a mammal of about 50 to 70 kg may contain between about 5 and 500 mg of the active ingredient. Typically, a dosage of the active compounds of the present invention, is a dosage that is sufficient to achieve the desired effect. Optimal dosing schedules can be calculated from measurements of agent accumulation in the body of a subject. In general, dosage may be given once or more daily, weekly, or monthly. Persons of ordinary skill in the art can easily determine optimum dosages, dosing methodologies and repetition rates.

To achieve the desired therapeutic effect, inhibitors or activators may be administered for multiple days at the therapeutically effective daily dose. Thus, therapeutically effective administration of compounds to treat a pertinent condition or disease described herein in a subject requires periodic (e.g., daily) administration that continues for a period ranging from three days to two weeks or longer. Typically, agents will be administered for at least three consecutive days, often for at least five consecutive days, more often for at least ten, and sometimes for 20, 30, 40 or more consecutive days. While consecutive daily doses are a preferred route to achieve a therapeutically effective dose, a therapeutically beneficial effect can be achieved even if the agents are not administered daily, so long as the administration is repeated frequently enough to maintain a therapeutically effective concentration of the agents in the subject. For example, one can administer the agents every other day, every third day, or, if higher dose ranges are employed and tolerated by the subject, once a week.

Optimum dosages, toxicity, and therapeutic efficacy of such compounds or agents may vary depending on the relative potency of individual compounds or agents and can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀. Agents that exhibit large therapeutic indices are preferred. While agents that exhibit toxic side effects can be used, care should be taken to design a delivery system that targets such agents to the site of affected tissue to minimize potential damage to normal tissues and, thereby, reduce side effects.

The data obtained from, for example, cell culture assays and animal studies can be used to formulate a dosage range for use in humans. The dosage of such compounds lies preferably within a range of circulating concentrations that include the ED₅₀ with little or no toxicity. The dosage can vary within this range depending upon the dosage form employed and the route of administration. For any agents used in the methods of the invention, the therapeutically effective dose can be estimated initially from cell culture assays. A dose can be formulated in animal models to achieve a circulating plasma concentration range that includes the IC₅₀ (the concentration of the agent that achieves a half-maximal inhibition of symptoms) as determined in cell culture. Such information can be used to more accurately determine useful doses in humans. Levels in plasma can be measured, for example, by high performance liquid chromatography (HPLC). In general, the dose equivalent of agents is from about 1 ng/kg to 100 mg/kg for a typical subject.

Exemplary dosages for an inhibitor of YAP or an activator of integrin β3 described herein are provided. Dosage for an inhibitor of YAP or an activator of integrin β3, for example, in the form of small organic compounds modulators, can be administered orally at between 5-1000 mg, or by intravenous infusion at between 10-500 mg/ml. Monoclonal antibody inhibitors/activators can be administered by intravenous injection or infusion at 50-500 mg/ml (over 120 minutes); 1-500 mg/kg (over 60 minutes); or 1-100 mg/kg (bolus) five times weekly. The inhibitors or activators can be administered subcutaneously at 10-500 mg; 0.1-500 mg/kg intravenously twice daily, or about 50 mg once weekly, or 25 mg twice weekly.

Pharmaceutical compositions of the present invention can be administered alone or in combination with at least one additional therapeutic compound. Exemplary advantageous therapeutic compounds include systemic and topical anti-inflammatories, pain relievers, anti-histamines, anesthetic compounds, and the like. The additional therapeutic compound can be administered at the same time as, or even in the same composition with, main active ingredient (e.g., an inhibitor of YAP or an activator of integrin β3). The additional therapeutic compound can also be administered separately, in a separate composition, or a different dosage form from the main active ingredient. Some doses of the main ingredient, such as an inhibitor of YAP or an activator of integrin β3, can be administered at the same time as the additional therapeutic compound, while others are administered separately, depending on the particular symptoms and characteristics of the individual.

The dosage of a pharmaceutical composition of the invention can be adjusted throughout treatment, depending on severity of symptoms, frequency of recurrence, and physiological response to the therapeutic regimen. Those of skill in the art commonly engage in such adjustments in therapeutic regimen.

### VI. Kits

The invention provides compositions and kits for practicing the methods described herein to prevent or treat a cardiovascular disease or inflammatory disease in a subject, which can be used for therapeutic purposes or as preventive measures.

Typically, the kits include a container containing a composition comprising an effective amount of a modulator of the Hippo-YAP pathway. For example, the composition may be a medicament for treating a cardiovascular disease or inflammatory disease, and it may be formulated for injection or oral ingestion. In other cases, the composition may be formulated as a dietary supplement, which may be ingested with food or beverage, by subjects who are at risk of a cardiovascular disease or inflammatory disease even though they may not have been diagnosed to actually suffer from the disease. Also, the kits often include a second composition, such as another known therapeutic agent effective for treating a cardiovascular disease or inflammatory disease, which can be used in combination with the first composition for enhanced effect. In addition, the kits of this invention may provide instruction manuals to guide users in the proper application of the composition(s) included therein.

### EXAMPLES

The following examples are provided by way of illustration only and not by way of limitation. Those of skill in the art will readily recognize a variety of non-critical parameters that could be changed or modified to yield essentially the same or similar results.

### Introduction

The Yorkie homologues YAP (Yes-associated protein) and TAZ (transcriptional coactivator with PDZ-binding motif, also known as WWTR1), effectors of the Hippo pathway have been identified as mediators for mechanical stimuli¹. However, the role of YAP/TAZ in haemodynamics-induced mechanotransduction and pathogenesis of atherosclerosis remains unclear. Here the present inventors show that endothelial YAP/TAZ activity is regulated by different patterns of blood flow, and YAP/TAZ inhibition suppresses inflammation and retards atherogenesis. Atheroprone-disturbed flow increases whereas atheroprotective unidirectional shear stress inhibits YAP/TAZ activity. Unidirectional shear stress activates integrin and promotes integrin-Gα₁₃ interaction, leading to RhoA inhibition and YAP phosphorylation and suppression. YAP/TAZ inhibition suppresses JNK signalling and downregulates pro-inflammatory genes expression, thereby reducing monocyte attachment and infiltration. In vivo endothelial-specific YAP overexpression exacerbates, while CRISPR/Cas9-mediated YAP knockdown in endothelium retards, plaque formation in ApoE^{-/-} mice. It is also shown that several existing anti-atherosclerotic agents such as statins inhibit YAP/TAZ transactivation. On the other hand, simvastatin fails to suppress constitutively active YAP/TAZ-induced pro-inflammatory gene expression in endothelial cells, indicating that YAP/TAZ inhibition could contribute to the anti-inflammatory effect of simvastatin. Furthermore, activation of integrin by oral administration of MnCl₂ reduces plaque formation. Taken together, these results indicate that integrin-Gα₁₃-RhoA-YAP pathway holds promise as a novel drug target against atherosclerosis.

### Results and Discussions

Endothelial cells (ECs) are constantly exposed to mechanical forces generated by blood flow. Different shear forces induce distinct cellular responses. Disturbed flow is associated with vascular inflammation and focal distribution of atherosclerotic lesions, while steady unidirectional shear stress (USS) is anti-inflammatory and atheroprotective².

The Hippo pathway, a newly identified kinase cascade, is involved in organ size control and tumour suppression. Activation of this pathway leads to inhibition of downstream effectors YAP/TAZ by promoting their phosphorylation and cytoplasmic retention³. YAP/TAZ were reported as sensors for mechanical stimuli including matrix stiffness, stretch and cell density¹. However, the role of YAP/TAZ in haemodynamics-mediated signal transduction and atherosclerosis is still unclear.

Indirect evidence implies possible involvement of YAP/TAZ in atherogenesis. The well-characterized YAP/TAZ target genes (CTGF and CYR61) are highly expressed in human atherosclerotic lesions⁴. Lysophosphatidic acid, a major atherogenic factor, is the potent activator of YAP and TAZ⁵. Statins, the widely used anti-atherosclerotic drugs, were identified as the strongest YAP inhibitors among 640 clinically used drugs⁶. However, direct evidence for YAP/TAZ activation in atherogenesis is still lacking.

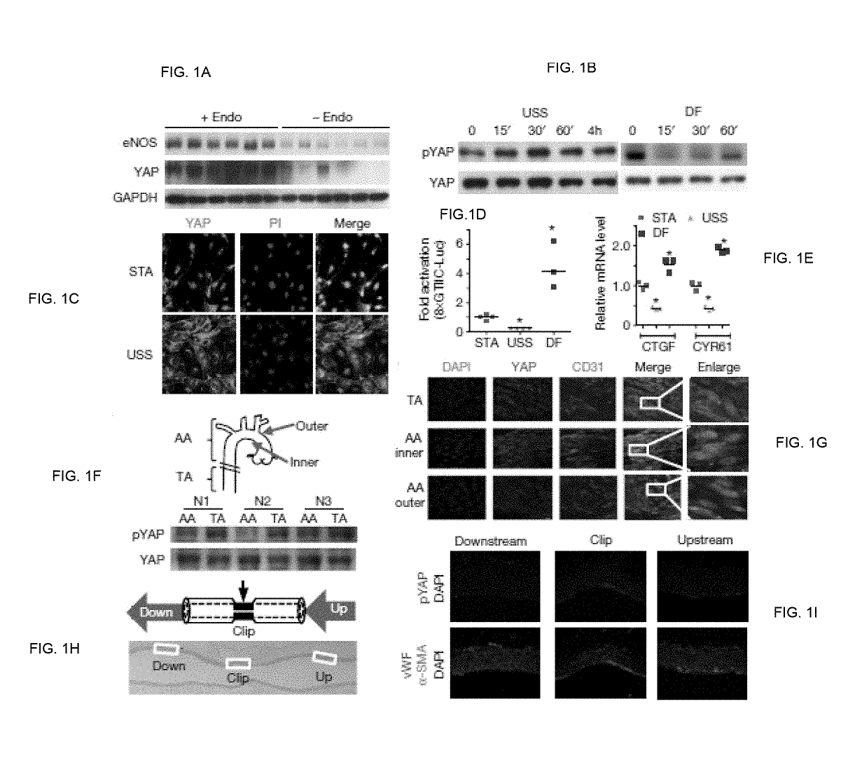

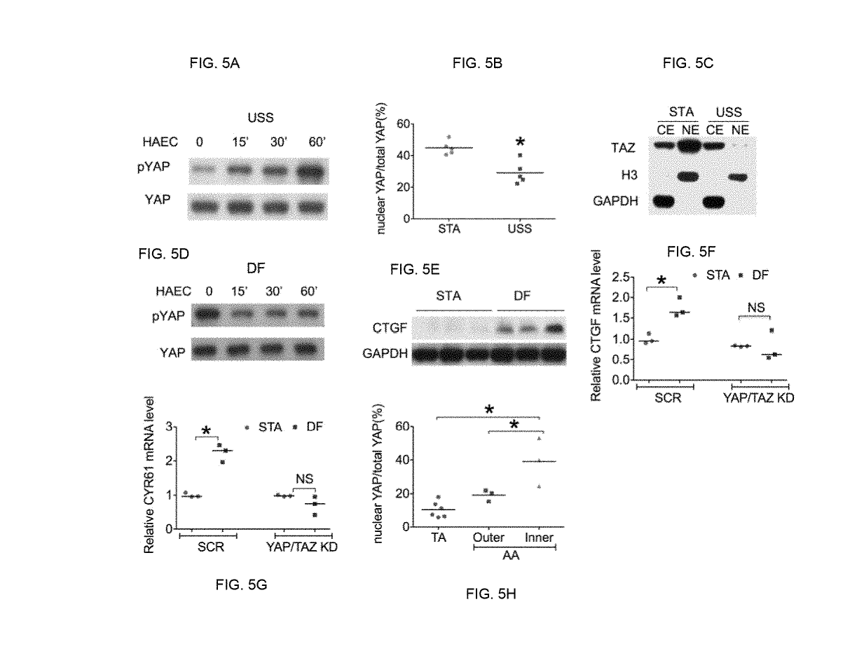

The present inventors first found mouse ECs express a higher level of YAP than other cells in aorta, indicating a possible role of YAP in maintaining endothelium homeostasis (FIG. 1A). To investigate the impact of haemodynamics on YAP activity, YAP phosphorylation (Ser127, pYAP) in human umbilical vein ECs (HUVECs) subjected to USS (12 dyn cm⁻²) or disturbed flow (0.5±6 dyn cm⁻², 1 Hz) was measured. Interestingly, USS inhibited, while disturbed flow activated, YAP activity. pYAP increased in HUVECs and human aortic ECs exposed to USS (FIG. 1B and FIG. 5A). Accordingly, increased YAP/TAZ cytoplasmic retention was observed in HUVECs subjected to USS (FIG. 1C and FIG. 5B, 5C). Congruently, USS suppressed transactivation activity of YAP/TAZ, indicated by reduced YAP/TAZ responsive luciferase (8× GTIIC-luc) reporter gene activity and downregulated expression of target genes (FIG. 1D, 1E). By contrast, disturbed flow reduced pYAP (FIG. 1B, and FIG. 5D), enhanced YAP/TAZ reporter gene activity (FIG. 1D) and increased YAP/TAZ target gene expression (FIG. 1E and FIG. 5E-5G). To investigate the effect of haemodynamics on YAP activity in vivo, the inventors determined YAP phosphorylation and nuclear localization in segments of mouse aorta and showed that pYAP level in aortic arch, an area exposed to disturbed flow, was lower than in thoracic aorta, an area exposed to USS (FIG. 1F). Consistently, in outer curvature of aortic arch and thoracic aorta, where blood flow is unidirectional, YAP was predominantly localized in the cytoplasm, while in the inner curvature of aortic arch, where blood flow is disturbed, YAP was mainly localized in the nuclei (FIG. 1G and FIG. 5H). Rat abdominal aorta cross-clamping is a model used to generate different flow patterns in vivo (FIG. 1H)⁷. The constricted region, where unidirectional flow is accelerated, exhibited highest pYAP levels. Modest pYAP levels were detected in the upstream region where blood flow is unidirectional, while low pYAP was observed in the downstream region where blood flow is disturbed (FIG. 1I).

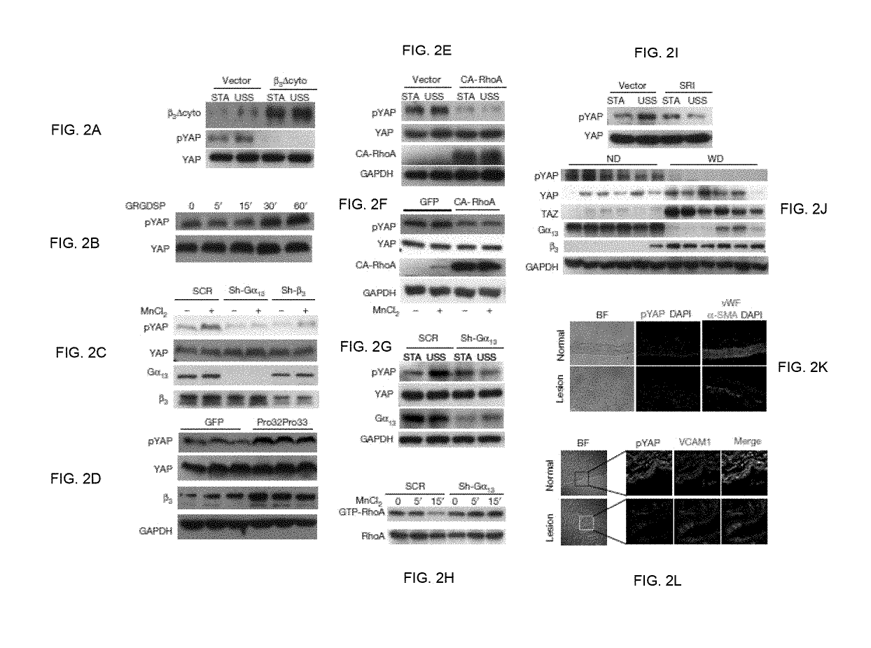

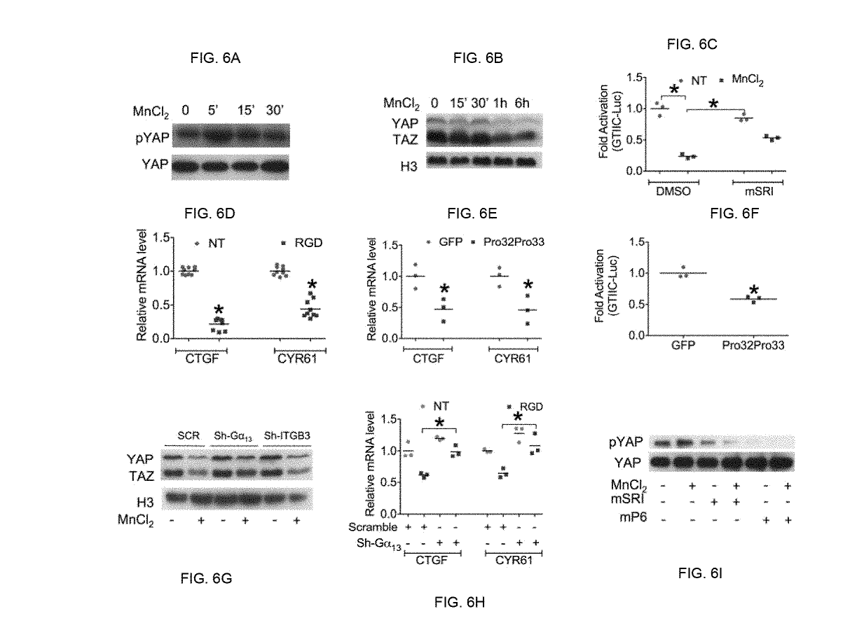

Integrin β₃ is a direct sensor for shear forces. The putative integrin agonists RGD-containing peptide (GRGDSP) or MnCl₂ can mimic the effect of USS⁸. To determine whether USS induces YAP phosphorylation through activating integrin, USS-induced YAP phosphorylation was examined in HUVECs transfected with loss-of-function mutation of integrin (with cytoplasmic domain deletion (β₃Δcyto))⁹. It was found β₃Δcyto overexpression abolished USS-induced YAP phosphorylation (FIG. 2A). Furthermore, treatment with GRGDSP or MnCl₂ increased pYAP in HUVECs (FIG. 2B and FIG. 6A). In addition, GRGDSP suppressed YAP/TAZ target gene expression (FIG. 6D). Congruently, MnCl₂ induced YAP/TAZ nuclear exportation (FIG. 6B) and reduced YAP/TAZ reporter gene activity (FIG. 6C), whereas integrin β₃ knockdown reversed MnCl₂-induced YAP phosphorylation (FIG. 2C). This evidence indicates that integrin activation directly induces YAP phosphorylation.

A previous study has suggested that flow-derived pulling force induces integrin activation by maintaining its extended conformation (ligand binding conformation)¹⁰. To test whether integrin β₃ in the extended conformation promotes YAP/TAZ phosphorylation, a Leu33Pro point mutation of integrin β₃ (Pro32Pro33 integrin) was constructed to mimic integrin β₃ activation¹¹. Indeed, Pro32Pro33 overexpression in HUVECs induced YAP phosphorylation (FIG. 2D), downregulated YAP/TAZ target gene expression (FIG. 6E) and suppressed YAP/TAZ reporter gene activity (FIG. 6F), indicating integrin β₃ mediates USS-induced YAP inhibition.

RhoA is one of the most important upstream activators of YAP/TAZ³. Integrin engagement and USS suppress RhoA activity^{8,12}. Therefore, it was hypothesized that RhoA mediates integrin-induced YAP/TAZ suppression. As expected, basal and USS- or MnCl₂-induced YAP phosphorylation was reduced in HUVECs transfected with constitutively active RhoA (Q63L) (CA-RhoA) (FIG. 2E, 2F).

G-protein subunit Gα₁₃ mediates integrin-induced RhoA suppression¹³⁻¹⁵. Therefore, the effect of Gα₁₃ knockdown was investigated in USS- or MnCl₂-induced RhoA inhibition and YAP phosphorylation. Neither USS nor MnCl₂ induced YAP phosphorylation when Gα₁₃ was silenced (FIG. 2C, 2G). Consistently, Gα₁₃ knockdown reduced MnCl₂-induced YAP nuclear exportation (FIG. 6G) and RhoA inhibition (FIG. 2H). Similarly, Gα₁₃ knockdown mitigated GRGDSP-induced suppression of YAP/TAZ target gene expression (FIG. 6H).

Physical interaction between integrin β₃ and Gα₁₃ induces RhoA inhibition^{13,14}. To understand whether integrin β₃ and Gα₁₃ interaction mediates YAP phosphorylation, two myristoylated cell-permeable short peptides, mSRI and mP6, which mimic the interaction domain of Gα₁₃ and integrin β₃ respectively, were used to selectively block association between Gα₁₃ and integrin β₃ (refs 13, 15). Similar to the effect of Gα₁₃ or integrin β₃ knockdown, mSRI or mP6 pretreatment abolished MnCl₂-induced suppression of YAP/TAZ reporter gene activity and YAP phosphorylation in HUVECs (FIG. 6C, 6I). Likewise, overexpression of SRI (Gα₁₃ blocking peptide) in HUVECs abolished USS-induced YAP phosphorylation (FIG. 2I).

Since haemodynamics is closely associated with pathogenesis of atherosclerosis, the expression of YAP, pYAP, TAZ, Gα₁₃ and integrin β₃ was compared in aortas of ApoE^{-/-} mice with or without Western-diet-induced atherosclerosis. The results showed downregulation of pYAP and Gα₁₃, and upregulation of TAZ, in aortas with atherosclerotic plaques (FIG. 2J). Consistent with a previous report¹⁶, the inventors found that integrin β₃ was highly expressed in mouse aortas with atherosclerosis, possibly because of compensatory response¹⁷. Immunofluorescence also showed that YAP phosphorylation reduced in lesion area of ApoE^{-/-} mice and in human atherosclerotic aortas (FIG. 2K, 2L). Taken together, these results reveal that integrin activation promotes integrin-Gα₁₃ association, which leads to RhoA suppression and subsequent YAP phosphorylation.

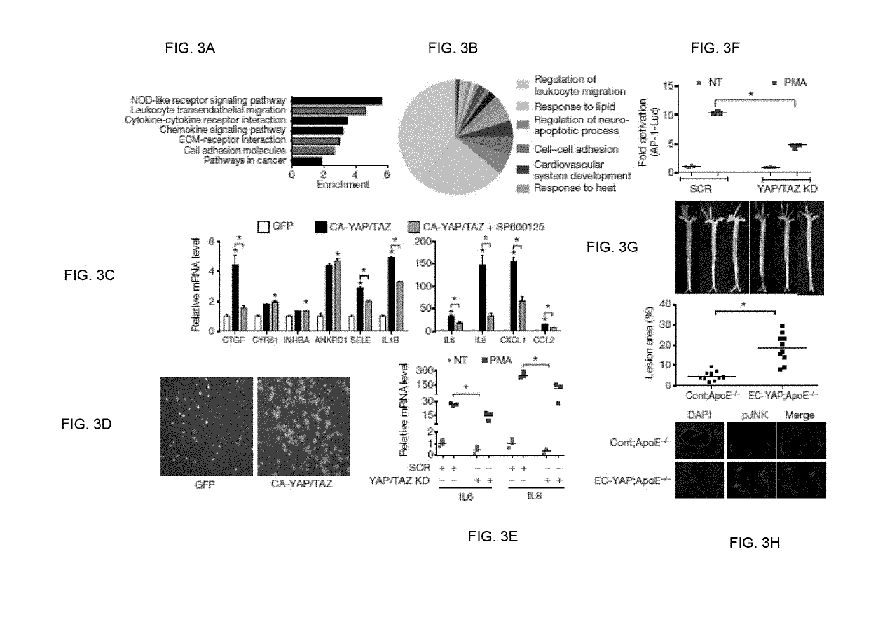

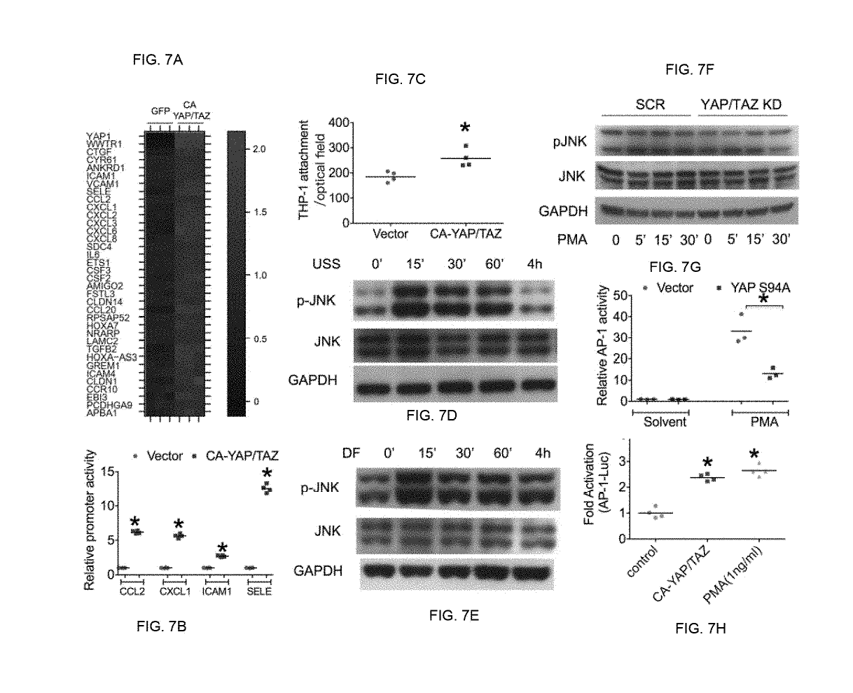

To explore the mechanism of YAP/TAZ activation in atherogenesis, the inventors analysed messenger RNA (mRNA) profiles in HUVECs transfected with constitutively active YAP (S127A) and TAZ (S89A) (CA-YAP/TAZ). Four hundred and sixteen differentially expressed genes were identified by RNA sequencing (RNA-seq) (P<0.05 and fold change cut-off>1.5). DAVID KEGG enrichment analysis¹⁸ revealed six enriched pathways (FIG. 3A), including ‘leukocyte transendothelial migration’, ‘ECM-receptor interaction’ and ‘cell adhesion molecules’, etc. Gene Ontology enrichment for biological process analysed by GlueGo¹⁹ indicated YAP/TAZ is associated with regulation of leukocyte migration (FIG. 3B). Indeed, the inventors showed more monocyte-endothelial adhesion associated with YAP/TAZ activation in HUVECs (FIG. 3D and FIG. 7C). Moreover, several pro-inflammatory markers, such as IL6, IL8 and SELE, were induced by YAP/TAZ activation (FIG. 3C and FIG. 7A). Promoter reporter assay showed that CA-YAP/TAZ induced expression of adhesion molecules by enhancing their transcription (FIG. 7B). However, deletion of the predicted TEAD binding sites, the known consensus DNA sequence for YAP-TEAD binding²⁰, in CXCLI and SELE promoters failed to reverse YAP/TAZ-induced reporter gene activity (data not shown), indicating other regulatory mechanisms might be involved. These results suggest that endothelial YAP/TAZ activation participates in the initiation of atherosclerosis by promoting monocyte adhesion.

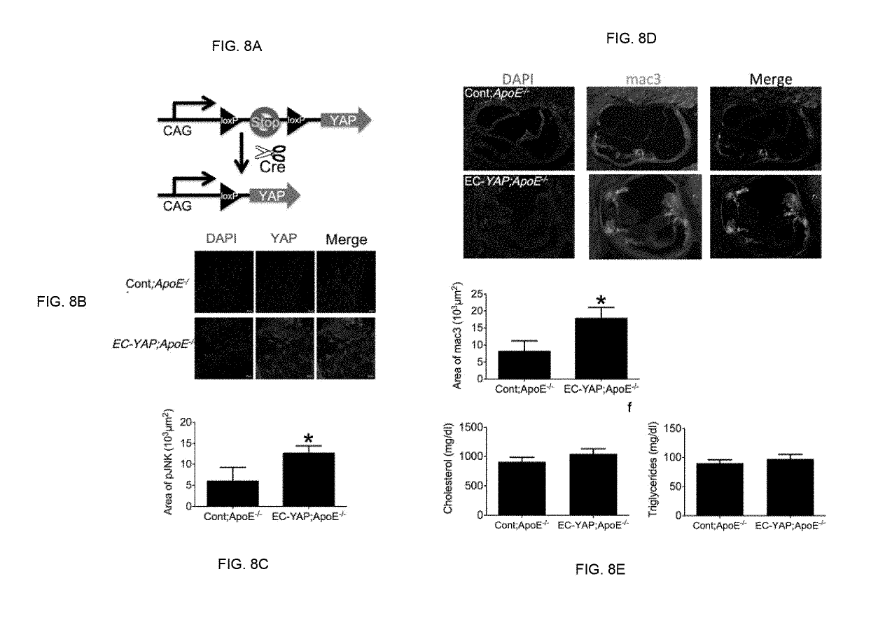

JNK is critical in atherogenesis²¹. USS inhibits tumour-necrosis factor (TNF)-α-induced JNK activation, while prolonged disturbed flow activates JNK^{22,23}. The results showed that both USS and disturbed flow transiently increased phospho-JNK. However, in contrast to sustained JNK phosphorylation in HUVECs exposed to disturbed flow, prolonged USS suppressed JNK phosphorylation (FIG. 7D, 7E). JNK effector activator protein (AP)-1 activity is reportedly increased by YAP/TAZ through JNK-YAP interaction²⁴⁻²⁶. It was therefore hypothesized that YAP/TAZ promotes endothelial activation through enhancing JNK activity. Indeed, JNK inhibitor SP600125 suppressed YAP/TAZ-induced pro-inflammatory gene expression (FIG. 3C). On the other hand, YAP/TAZ knockdown reduced basal and phorbol ester (PMA)-induced phospho-JNK, expression of JNK target genes IL6 and IL8 as well as AP-1 reporter gene activity (FIG. 3E, 3F and FIG. 7F)²⁷. Dominant negative YAP (YAP S94A) suppressed PMA-induced AP-1 reporter gene activity, whereas CA-YAP/TAZ enhanced AP-1 reporter gene activity (FIG. 7G, 7H). To assess whether YAP activates JNK and accelerates atherosclerotic plaque formation in vivo, the inventors generated EC-specific YAP overexpression mice on ApoE^{-/-} background (Tie2^{Cre/+}; YAP-COE^{g/+}ApoE^{-/-} (EC-YAP; ApoE^{-/-})) (FIG. 8A, 8B). After 4 weeks of feeding on Western diet, EC-YAP; ApoE^{-/-} mice showed significantly increased plaque formation (FIG. 3G), accompanied by increased expression of p-JNK and macrophage marker Mac3 compared with control littermates (Cont;ApoE^{-/-}) (FIG. 3H and FIG. 8C, 8D). Similar total cholesterol and triglyceride levels suggested that the atherogenic effect of endothelial YAP is unlikely to be related to lipid metabolism (FIG. 8E, 8F).

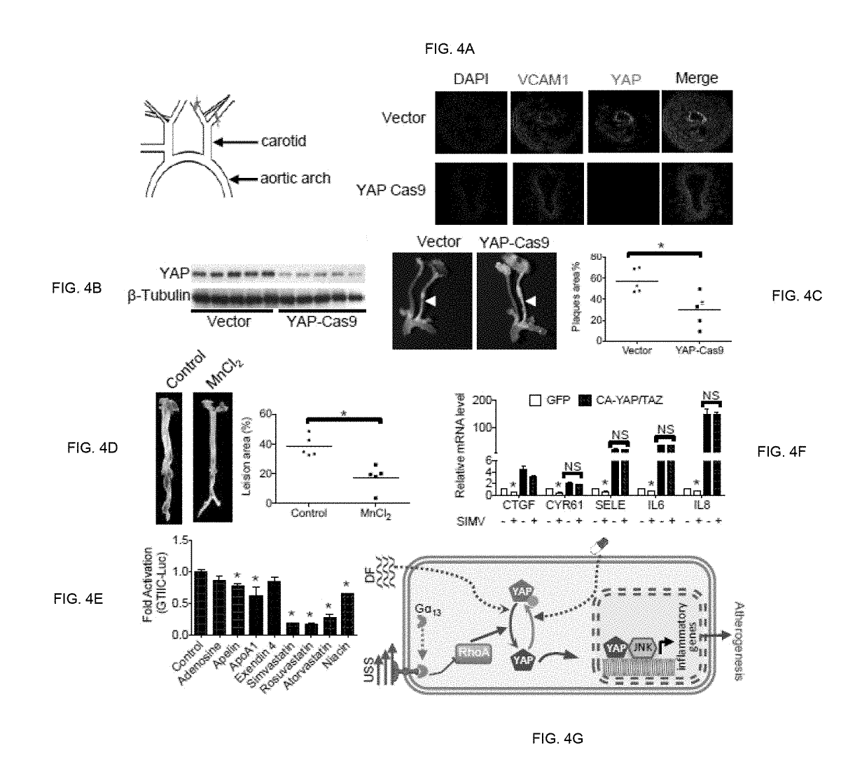

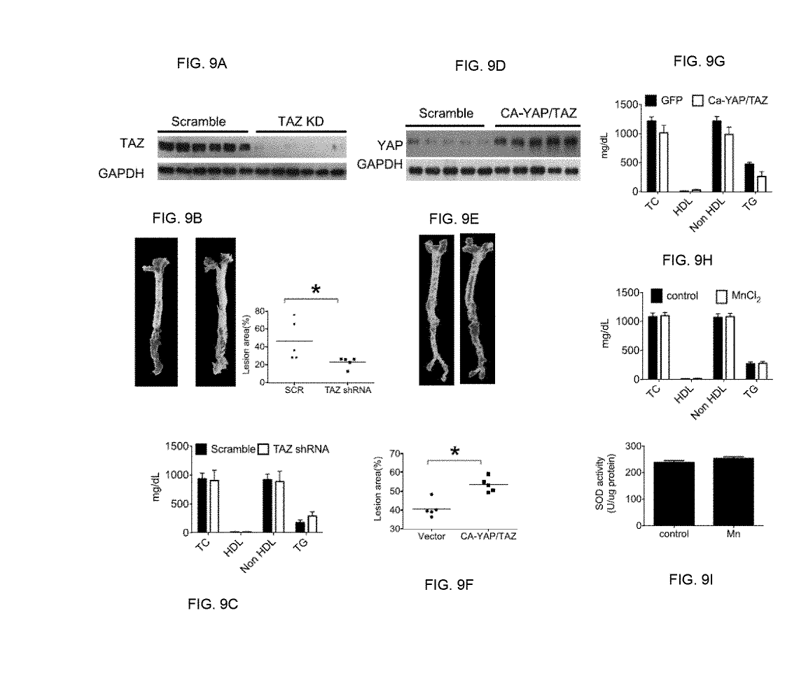

To demonstrate that disturbed flow-associated atherosclerosis is mediated by endothelial YAP activation in vivo, ApoE^{-/-} mice received partial ligation surgery on the left carotid artery to develop disturbed flow-enhanced atherosclerosis. EC-specific Yap knockdown was achieved by using EC-enhanced AAV-mediated CRISPR/Cas9 (ref 28) genome-editing system controlled by EC-specific ICAM2 promoter. Immunohistochemistry and western blotting showed efficient YAP knockdown in ECs (FIG. 4A, 4B). Three weeks after surgery, severe plaques developed in control ApoE^{-/-} mice. However, mice with EC-specific YAP knockdown exhibited reduced plaque formation (FIG. 4C). Mice injected with adenovirus-mediated TAZ short hairpin RNA (shRNA) also showed delayed atherogenesis (FIG. 9A-9C). Furthermore, oral administration of MnCl₂ reduced plaque formation in ApoE^{-/-} mice on Western diet for 12 weeks (FIG. 4D), without affecting lipid profile or superoxide dismutase activity (FIG. 9H, 9I). Conversely, plaque formation increased in mice injected with AAV expressing CA-YAP/TAZ (FIG. 9D-9F). In summary, both gain- and loss-of-function experiments in vivo show the importance of YAP/TAZ activation in atherogenesis.

To examine whether existing anti-atherosclerotic drugs inhibit YAP/TAZ activity, several compounds were tested (Table 1). In addition to statins, which inhibit YAP/TAZ in tumour cells⁶, apelin, ApoA1 and niacin also suppressed YAP/TAZ activity (FIG. 4E). To understand whether YAP/TAZ suppression contributes to the anti-inflammatory effect of statin, HUVECs were transfected with CA-YAP/TAZ. Compared with HUVECs transfected with vector control, simvastatin failed to suppress expression of pro-inflammatory genes induced by CA-YAP/TAZ, suggesting YAP/TAZ inhibition might be involved in anti-inflammatory and anti-atherogenic effect of statins (FIG. 4F).

In summary, this study provides novel evidence showing that endothelial YAP/TAZ activation induced by atheroprone-disturbed flow promotes inflammation and atherogenesis by enhancing JNK activity, whereas the atheroprotective USS inhibits YAP/TAZ by modulating the integrin-Gα₁₃-RhoA pathway (FIG. 4G). Endothelial YAP/TAZ knockdown or MnCl₂ treatment delays atherogenesis, indicating YAP/TAZ could become a potential therapeutic target against atherosclerosis, as demonstrated by the YAP/TAZ-inhibitory effect of several anti-atherosclerotic drugs, especially statins.

### Materials and Methods

### Antibodies

The antibodies used for western blotting included anti-YAP/TAZ (1:1,000; 8418; Cell Signaling Technology, USA), anti-YAP (1:1,000; Cell Signaling Technology, USA), anti-pYAP (1:1,000; Ser 127, 4911S; Cell Signaling Technology, USA), anti-TAZ (1:1,000; ab84927; Abcam, UK), anti-JNK (1:1,000; 9252h; Cell Signaling Technology, USA), anti-pJNK (1:1,000; 9255; Cell Signaling Technology, USA), anti-CTGF (1:1,000; ab6992; Abcam, UK), anti-Gα₁₃ (1:1,000; ab128900; Abcam, UK), anti-integrin β₃ (1:1,000; 4702; Cell Signaling Technology, USA), anti-RhoA (1:1,000; ab54835; Abcam, UK) and anti-eNOS (1:1,000; BD Biosciences, USA).

The antibodies used for immunostaining included anti-pYAP (1:100; Ser 127, 4911S; Cell Signaling Technology, USA), anti-YAP (1:100; Cell Signaling Technology, USA) and anti-pJNK (1:100; 9255; Cell Signaling Technology, USA).

### Quantitative Real-Time PCR

RNA was extracted by using TRIzol Reagent (Thermo) according to the manufacturer's protocol. cDNA was synthesized using a High-Capacity cDNA Reverse Transcription Kit (Thermo). Quantitative PCR was performed using SYBR Select (Thermo) following the manufacturer's protocol. GAPDH was used as the internal control. Primers used for quantitative real-time PCR were included in Table 2.

### Western Blotting

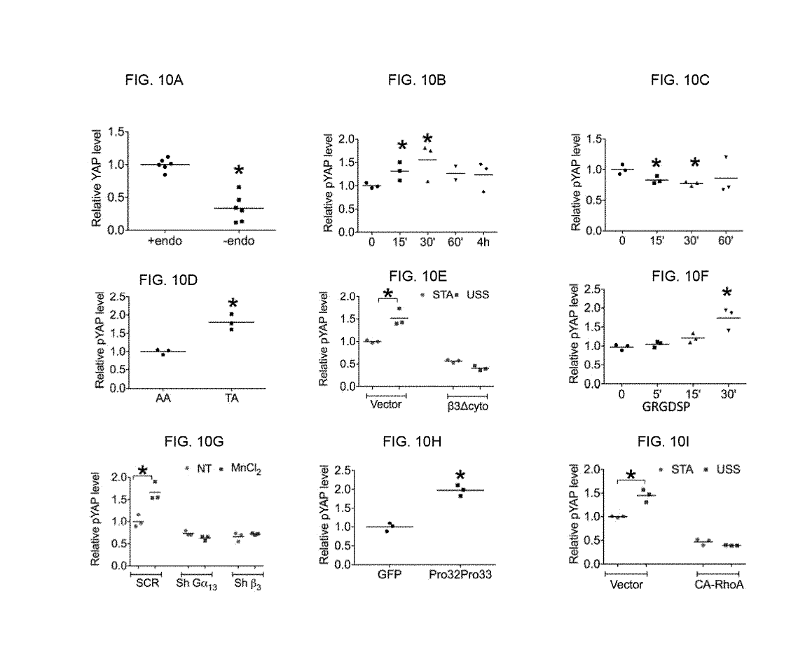

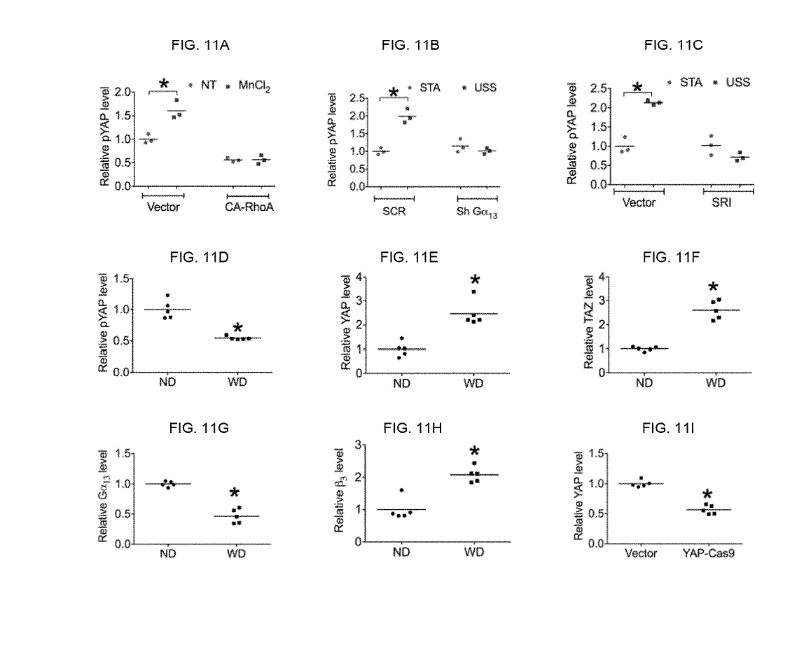

Cells or tissues were homogenized in cold RIPA lysis buffer supplemented with complete protease inhibitors cocktail and phosSTOP phosphatase inhibitor (Roche). The protein concentration was determined using Bradford Assay (Bio-Rad). Ten micrograms of protein were resolved by SDS-polyacrylamide gel electrophoresis and transferred to the PVDF membrane (Bio-Rad). Target protein was detected using specific primary antibody. Bound antibodies were detected by horseradish-peroxidase-conjugated secondary antibody and visualized by enhanced chemiluminescence (Cell Signaling Technology). Experiments were repeated three times and the target protein level was quantified by imageJ and normalized to internal control (or pYAP was normalized by total YAP) (FIGS. 10 and 11).

### Cell Culture

HUVECs and human aortic ECs were purchased from Lonza (EGM, Clonetics, Lonza, Walkersville, Md., USA). Lonza guarantees that the cells express CD31/105, von Williebrand Factor VIII, and are positive for acetyated low density lipoprotein uptake. Mycoplasma contamination was not tested during the experiments. HUVECs maintained in EGM supplemented with EGS and FBS at 37° C. in an incubator with 95% humidified air and 5% CO₂ and passed every 3 days. Cells within seven passages were used in in vitro study.

### GST-RBD Pull-Down for Active RhoA Detection

GST-RBD recombinant protein was purified from BL21 (DE3) Escherichia coli and affinity conjugated to glutathione sepharose beads (Pharmacia). For GST affinity pull-down, 10⁷ cells were lysed in 1 ml Weak Lysis Buffer (Beyotime) supplemented with protease inhibitors (Roche). Cell lysates were centrifuged at 15,000 g at 4° C. for 20 min to remove cell debris. Cell lysates were incubated in sepharose beads conjugated with 1 μg GST-RBD and incubate at 4° C. for 2 h with constant agitation, and precipitated by centrifugation at 1,000 r.p.m. for 10 min. After three washes, beads were collected by centrifugation and boiled in 2× SDS loading buffer for 5 min. The active RhoA was determined by western blotting.

### Experimental Animals

Animals were supplied by the University Laboratory Animal Services Centre and approved by the Ethical Committee of Animal Research (CUHK). The animals used in the present study included Sprague-Dawley rats, apolipoprotein E deficient (ApoE^{-/-}) mice and EC-specific YAP overexpression transgenic mice. The animals were kept at a constant temperature (21±1° C.) under 12/12-h light/dark cycle and had free access to water and standard chow unless specified.

### Construction of EC-Specific YAP Overexpression Mice

CAG loxp-stop-loxp-YAP mice were generated in a C57BL/6 background in Model Animal Research Center (Nanjing, China). YAP-COE mice were crossed with ApoE^{-/-} mice and then Tie-2-Cre^{-/-} mice. The 6-week-old ApoE^{-/-};YAP-COE;Tie-2-Cre^{+/-} and ApoE^{-/-}; YAP-COE; Tie-2-Cre^{+/-} mice were bred and housed in temperature-controlled cages under a 12/12-h light/dark cycle with free access to water in Tianjin Medical University Animal Center. Study protocols and the use of animals were approved by the Institutional Animal Care and Use Committee of Tianjin Medical University (Tianjin, China). The mice were fed a Western diet (Research Diets, D12109) containing 40 kcal% fat, 1.25% cholesterol and 0.5% cholic acid for 4 weeks before being killed. Aortas were isolated to assess lesion formation and distribution by Oil Red O staining. Aortic roots were stained for pJNK, α-SMA and macrophage.

En face Staining

Mouse aortas were fixed with 4% paraformaldehyde for 15 min. After permeabilization/blocking in 0.05% Triton X-100 (in PBS) and 1% BSA and for 0.5 h at room temperature, aortas were incubated at 4° C. overnight in incubation buffer containing 1% BSA and the primary antibody including YAP1 (Abcam, ab52771), CD31 (Abcam, ab24590). After being washed in PBS three times, aortas were incubated with Alexa-Fluor 488-, Alexa-Fluor 594-conjugated secondary antibodies (ZSGB-BIO, Beijing) for 1 h at room temperature. The fluorescent signal was detected by a Leica confocal laser scanning microscopy.

### Disturbed Flow In Vivo

Stenosis of the abdominal aorta of rats was induced using a U-shaped titanium clip, as described^{29,30}. Briefly, after anaesthetization with isoflurane, the rat was laid supine and a lower midline abdomen incision was made; the part of the intestine was gently lifted out of the abdominal cavity and kept moist with saline throughout the surgical procedure. The aorta, left and right common iliac artery were exposed and the accompanying vein was carefully separated. The clip was held with a pair of forceps and placed around the isolated segment (1 cm from the arterial bifurcation) to partly constrict the abdominal aorta. The extent of clipping was controlled by placing a stopper of given size between the two arms of the forceps. Two weeks later, the rat was euthanized by intoxication with 100% carbon dioxide, and the aorta was perfusion-fixed with 4% (w/v) paraformaldehyde at 120 mm Hg. The fixed aorta was embedded in paraffin blocks for immunohistochemical staining.

Partial ligation of carotid artery was generated as described before'. Briefly, ApoE^{-/-} mice were anaesthetized by intraperitoneal injection of xylazine (10 mg/kg) and ketamine (80 mg/kg) mixture. A ventral midline incision (4-5 mm) was made in the neck. Left carotid artery was exposed by ventral midline incision (4-5 mm) in the neck. Left external carotid, internal carotid and occipital arteries were ligated, while the superior thyroid artery was left intact. Mice were monitored until recovery in a chamber on a heating pad after surgery and fed the Western diet immediately after surgery until killed.

### Immunohistochemical Staining

Immunohistochemical staining was performed on serial sections (5 μm thick) of paraffin-embedded rat abdominal aortas and ApoE^{-/-} mouse aortas using pYAP (Cell Signaling), EC- and SMC-specific markers (that is, vWF and α-SMA, respectively) (Merck Millipore). Briefly, the sections were de-waxed in xylene, rehydrated in descending grades of alcohol and permeabilized by incubating for 10 min in sodium citrate for 10 min at 95° C. Sections were cooled down to room temperature and blocked with blocking reagent (Merck Millipore) for 30 min. One section was incubated with antibody against pYAP (1:100) overnight at 4° C., followed by Alexa Fluor 594-conjugated goat anti-rabbit IgG (1:1,000; Invitrogen) secondary antibody in blocking reagent for 1 h at room temperature. The secondary section was incubated with antibodies against vWF and a-SMA (1:100 each) overnight at 4° C., respectively, followed by Alexa Fluor 594-conjugated goat anti-rabbit IgG and Alexa Fluor 488-conjugated goat anti-mouse IgG (1:1,000; Invitrogen) secondary antibodies in blocking reagent for 1 h at room temperature. Nuclei were co-stained by DAPI (Invitrogen) in PBS for 5 min. The sections were spin-dried and mounted with ProLong Gold (Invitrogen) on glass coverslips. Images were acquired and analysed using a Zeiss fluorescence microscope with Axiovision image analysis software.

Oral Administration of MnCl₂ in ApoE^{-/-} Mice

ApoE^{-/-} mice (male, 12 weeks old) were fed a Western diet, and MnCl₂ was administered through voluntary water consumption. Water consumption rate was predetermined by monitoring the volume of water remained. MnCl₂ was supplemented to drinking water to achieve 5 mg/kg body weight. Mice body weight and water consumption were adjusted weekly to adapt to the change of body weight and water consumption. After feeding on the Western diet for 3 months, the mice were killed and the atherosclerotic plaque formation was determined by Oil Red O staining.

### Oil Red O Staining for Atherosclerotic Plaques in Mouse Aorta

The ApoE^{-/-} mice were killed by CO₂ asphyxiation. Mouse aortas were dissected in cold PBS and cut open to expose the atherosclerotic plaques. After fixation in 4% formaldehyde for 16 h at 4° C., the tissues were first rinsed in water for 10 min and then in 60% isopropanol. The aortas were stained with Oil Red O for 15 min with gentle shaking, and rinsed again in 60% isopropanol and then in water for three rinses. The samples were fixed on the cover slides with the endothelial surface facing upwards. The images were recorded using an HP Scanjet G4050. The plaque areas were determined using National Institutes of Health ImageJ software and calculated by expressing the plaque area relative to the total vascular area.

### Human Aortic Specimens

The experiments were approved by the Hospital Human Subjects Review Committee (IRB approval number TSGHIRB 2-103-05-132) of Tri-Service General Hospital in Taipei and were conducted under the guidelines established by the Ethics Review Board of National Health Research Institutes, Taiwan. Written informed consent was obtained from all individuals. Human aortic tissue specimens were from patients with acute type-A aortic dissection. These samples were collected during emergency aortic surgery. The diseased segments of aorta (that is, dissecting aortic aneurysm) in these patients were all resected and replaced by an artificial inter-position graft, respectively. Specimens were fixed in paraformaldehyde, paraffin-embedded and cut into 5μm sections. YAP Ser127 phosphorylation was determined by immunofluorescence imaging.

### RNA-Sequencing

HUVECs were transfected with pWCXIH-Flag-YAP-S127A (a gift from K. Guan, Addgene 33092) and 3× Flag pCMV5-TOPO TAZ (S89A) (a gift from J. Wrana, Addgene 24815) or pEGFP-N1 by Neon transfection system (Invitrogen, USA)^{32,33}. Four hours after transfection, cells were harvested and RNA was extracted using RNeasy Mini Kit (Qiagen, Germany). The extracted RNA samples were sent to Beijing Genomics Institute (BGI) for RNA-sequencing analysis. P<0.05 and fold change>1.5 was used as a threshold for different regulated genes. DAVID tools were used for the pathways enrichment analysis and GlueGo was used for the Gene Ontology analysis.

### Haemodynamics Study In Vitro

Ibidi flow system (MIDI, Germany) was used to generate USS and disturbed flow (12 dyn cm' for USS and 0.5±6 dyn cm⁻², 1 Hz for disturbed flow). μ-slide I 0.4 Luers (IBIDI, LLC) was used for immunofluorescence studies. The slide was coated with 50 μg/mL fibronectin for 24 h. Seven thousand HUVECs were seeded onto the slide. After cells were adapted to medium containing 2% FBS (10% fatty acid free BSA for disturbed flow) for 6 h, the slides were mounted onto the Ibidi flow system. For immunostaining of USS-induced YAP/TAZ nuclear exportation, cells were subjected to USS for 6 h. For western blotting and reverse transcription real-time PCR analysis, the μ-slides were replaced with a custom-built flow chamber, which could accommodate more cells. Glass slides (75 mm×38 mm; Corning) were coated with fibronectin (50 μg/mL). HUVECs were seeded on slides and allowed to attach on the bottom for 16 h. For USS, the medium was replaced with EGM supplemented with 2% FBS for 6 h. For disturbed flow, cells were incubated in EGM supplemented with 10% fatty acid free BSA (Sigma). The slides were mounted onto the flow chamber and connected to the Ibidi flow system. The cells were then subjected to USS or disturbed flow. For USS-induced YAP phosphorylation, 15 min of shear force was applied unless otherwise noted. For USS-induced YAP translocation, 6 h of shear force was applied. For reverse transcription real-time PCR analysis, 4 h of shear stress was sufficient to inhibit the expression of YAP/TAZ target genes. For reporter gene assay, 48 h of shear forces were applied to HUVECs.

### Plasmid Construction

To construct the reporter plasmids for adhesion molecules, human genomic DNA was purified from HUVECs using a Universal Genomic DNA Extraction Kit Ver 3.0 (Takara, Japan). The promoters of ICAM1, E-Selectin, MCP1 and CXCL1 were PCR amplified from human genomic DNA using the primers listed in Table 2. A 2.1 kb fragment (−1784 to +328) from the ICAM1 promoter, a 2.2 kb fragment (−1807 to +475) from the E-Selectin promoter, a 4 kb fragment (−3992 to +73) from MCP1 promoter and a 1.3 kb fragment (−1256 to +84) from CXCL1 promoter were amplified. The PCR products were gel purified by gel extraction kit (Takara, Japan) and digested with restriction enzymes. The digested fragments were gel purified and ligated to pGL3 reporter plasmid digested by corresponding restriction enzymes. The ligation products were then heat inactivated at 65° C. for 15 min and transformed into the DH5α competent cells.

The Pro32Pro33 integrin was derived from pcDNA3.1-beta-3 (a gift from T. Springer, Addgene plasmid 27289) by point mutation³⁴.

Primers used for plasmids construction were included in Table 2.

### Adenovirus Production

To generate the adenovirus shuttle vector pShuttle-U6, the U6 promoter and 1.9 kb stuffer sequence was excised from pLKO.1 (a gift from D. Root, Addgene plasmid 10878) with NotI/XhoI and ligated into pShuttle plasmid pre-digested with restriction enzymes accordingly. Short hairpin RNA targeting mouse TAZ was generated using a protocol similar to pLKO.1 shRNA plasmids (Addgene) construction protocol. TAZ shRNA sequence, TRCN0000095951, which was validated by Mission shRNA (Sigma Aldrich), was used to generate shuttle plasmids for TAZ shRNA.

Recombinant adenovirus was generated using the AdEasy system³⁵. Briefly, pShuttle-U6 vector containing shRNA was digested with Pmel and co-transformed with adenoviral backbone plasmid pAdEasy-1 for homologous recombination in E. coli BJ5183 cells. Positive recombinants were linearized by PacI digestion and transfected into HEK-293A cells for virus packaging. The medium and cells were collected until the cytopathic effect was apparent. After three cycles of freeze and thaw to release the virus, the cell debris was removed by centrifugation at 3,000 r.p.m. for 15 min. The virus-containing supernatant was collected by PEG precipitation, followed by dialysis against saline with 100K MWCO dialysis tubing (Spectrum Labs).

### Lentivirus Production

Lentiviral shuttle plasmids for YAP (TRCN0000300325), TAZ (TRCN0000095951), Gα₁₃ (TRCN0000036885) and integrin β3 (TRCN0000003236) shRNA were purchased from Sigma. Plasmid cocktail containing 1 μg of resultant shuttle plasmid, 750 ng of psPAX2 packaging plasmid and 250 ng of pMD2.G envelope plasmid were co-transfected to HEK-293FT cells. The medium was changed 15 h after transfection; 48 and 72 h after transfection, the medium containing the lentiviral particles was harvested then passed through 0.45 μm filters to remove cell debris. The virus was precipitated with PEG and suspended in PBS containing 4% sucrose. The lentiviral solutions were then aliquoted to vials and stored at −80° C.

### Construction of AAV Shuttling Plasmid for CA-YAP/TAZ Overexpression

YAP1 S127A was amplified from pWCXIH-Flag-YAP-5127A (a gift from K. Guan, Addgene 33092) and ligated to pAAV-MCS (Stratagene) to generate the pAAV-YAP1 S127A shuttle plasmid. A similar strategy was used to generate the pAAV-TAZ S89A from 3× Flag pCMV5-TOPO TAZ (S89A) (a gift from J. Wrana, Addgene 24815).

### Construction of Endothelial Specific AAV-Mediated CRISPR/Cas9 Shuttle Plasmid

pX601-AAV-CMV: NLS-SaCas9-NLS-3×HA-bGHpA;U6::BsaI-sgRNA (a gift from F. Zhang, Addgene plasmid 61591) was used to generate the EC-specific Cas9 for YAP in vivo genome editing²⁸. Three sgRNA sequences for YAP were predicted by CCTop (CRISPR/Cas9 target online predictor)³⁶. ICAM2 endothelium-specific promoter from human was synthesized by GenScript and replaced the CMV promoter in pX601-AAV-CMV¹⁴.

Primers used for sgRNA were included in the Table 2.

### Endothelial Enhanced AAV Packaging

The shuttle plasmids were co-transfected into HEK-293T with endothelial enhanced RGDLRVS-AAV9-cap plasmid (provided by O. J. Muller, Universitat Heidelberg, Germany) and pHelper plamid (Stratagene)³⁷. After co-transfection for 72 h, the AAV viral particles were isolated according to the protocol reported in ref 38. Briefly, the cells were harvested and re-suspended in 1× restore buffer and the nuclei were extracted by homogenization. Viral particles were extracted by using nuclear lysis buffer. The viral particles were purified by PEG concentration, followed by dialysis against saline with 100K MWCO dialysis tubing (Spectrum Labs) to remove impurities, and concentrated. The viral titration was determined by qPCR and adjusted to 10¹⁰ plaque-forming units per millilitre in PBS containing 4% sucrose.

### Virus Administration

For adenovirus-mediated Taz shRNA, viruses (10⁹ plaque-forming units) were administered to ApoE^{-/-} mice (male, 12 weeks old) that had been fed on Western diet (Research Diets) for 4 weeks through tail vein injection. The mice were then fed on Western diet for 2 more months. The atherosclerotic plaque formation was visualized by Oil Red O staining. For AAV-mediated CA-YAP/TAZ overexpression and YAP-Cas9, the viruses (10⁹ plaque-forming units) were administrated to ApoE^{-/-} mice (male, 12 weeks old) through tail vein injection before feeding on Western diet or receiving the carotid partial ligation surgery.

### Statistical Analysis

Statistics analyses were performed using GraphPad Prism 5.0. The sample sizes were not predetermined by statistical methods. The samples were not randomized and the investigators were not blinded to allocation during experiments and outcome assessment. At least three independent experiments were performed for all biochemical experiments and the representative images were shown. Results represent mean±s.e.m. Student's t-test (unpaired two-tailed) was used in the analysis. No samples, mice or data points were excluded from the reported analysis. Levels of probabilities less than 0.05 were regarded as significant.

### Data Availability

The RNA-seq data that support the findings of this study have been deposited in BioSamples database (website: ebi.ac.uk/biosamples) under accession number SAMN04565728.

TABLE 1
Drugs and concentrations used for the YAP/TAZ inhibition test
Extended Data Table 1 | Drugs and concentrations
used for the YAP/TAZ inhibition test

| | | | |
|---|---|---|---|
| | Stock | Working | |
| Drugs | concentration | concentration | Company |

| | | | | | |
|---|---|---|---|---|---|
| Adenosine | 10 | mM | 10 | μM | Sigma |
| Apelin | 1 | mM | 100 | nM | Sigma |
| Exendin 4 | 100 | μM | 10 | nM | Sigma |
| Nicotinic acid | 500 | mM | 3 to 5 | mM | Sigma |
| (VB3 niacin) | | | | | |
| ApoA1 | 1 | mg/mL | 10 | μg/mL | Sigma |
| Rosuvastatin | 10 | mM | 10 | μM | Cayman |
| Simvastatin | 100 | mM | 1 | μM | Cayman |
| Atorvastatin | 10 | mM | 1 | μM | Cayman |

TABLE 2
Primers used in this study

| | | |
|---|---|---|
| Gene | Forward | Reverse |

Promoter for cloning

| | | |
|---|---|---|
| ICAM1 | CTCAGAAAGTGACCCGCCAT | CCTCCATCTCCAACCCCCTA |
| SELE | CGTTCAGGTCTGCTGACAGT | TTTTGTGACTGCCACCCACT |
| CCL2 | GGGCTGGCTCAGAAGACAAT | GGAGCTGGATTTGGGGTTCA |
| CXCL1 | GGTCTCCATTGGGTCAATGCT | GGGGACTTCACGTTCACACT |

P32P33 mutation

| | | |
|---|---|---|
| P32P33 | CCTGCCTCCGGGCTCACCTC | AGCCCGGAGGCAGGGCCTC |

Primers for YAP-Cas9

| | | |
|---|---|---|
| Cas9-YAP-1 | CACCGTCTGAGGCACGTTGGCCGTCT | AAACAGACGGCCAACGTGCCTCAGAC |
| Cas9-YAP-2 | CACCGTGCACGACCTGGTGGCCGGCC | AAACGGCCGGCCACCAGGTCGTGCAC |
| Cas9-YAP-3 | CACCGCCAGAGACAACGCCACTGGCT | AAACAGCCAGTGGCGTTGTCTCTGGC |

Real-time PCR primers

| | | |
|---|---|---|
| rt-hum-CTGF | ACCGACTGGAAGACACGTTTG | CCAGGTCAGCTTCGCAAGG |
| rt-hum-CYR61 | TGAAGCGGCTCCCTGTTTT | CGGGTTTCTTTCACAAGGCG |
| rt-hum-VCAM1 | CAGTAAGGCAGGCTGTAAAAGA | TGGAGCTGGTAGACCCTCG |
| rt-hum-ICAM1 | TTGGGCATAGAGACCCCGTT | GCACATTGCTCAGTTCATACACC |
| rt-hum-SELE | TGTGGGTCTGGGTAGGAACC | AGCTGTGTAGCATAGGGCAAG |
| rt-hum-CCL2 | CAGCCAGATGCAATCAATGCC | TGGAATCCTGAACCCACTTCT |
| rt-hum-INTBA | ACGGGTATGTGGAGATAGAGGA | GGACTTTTAGGAAGAGCCAGACT |
| rt-hum-ANKRD1 | AGAACTGTGCTGGGAAGACG | GCCATGCCTTCAAAATGCCA |
| rt-hum-IL1B | CTTCGAGGCACAAGGCACAA | TTCACTGGCGAGCTCAGGTA |
| rt-hum-IL6 | CTCAATATTAGAGTCTCAACCCCCA | GAGAAGGCAACTGGACCGAA |
| rt-hum-IL8 | CCACCGGAGCACTCCATAAG | GATGGTTCCTTCCGGTGGTT |
| rt-hum-CXCL2 | TGTGACGGCAGGGAAATGTA | TCTGCTCTAACACAGAGGGAAAC |
| rt-hum-GAPDH | ACGGATTTGGTCGTATTGGGC | TTGACGGTGCCATGGAATTTG |
| rt-hum-LATS1 | CAGGGATACTTGGGGTTGCT | AGGAAGTCCCCAGGACTGTTA |
| rt-hum-TAZ | ATCCCCAACAGACCCGTTTC | GAACGCAGGCTTGCAGAAAA |
| rt-hum-YAP1 | GCTACAGTGTCCCTCGAACC | CCGGTGCATGTGTCTCCTTA |
| rt-mus-Ctgf | CCCTGCCCTAGCTGCCTACCG | GCTTCGCAGGGCCTGACCAT |
| rt-mus-Cyr61 | GCCGTGGGCTGCATTCCTCT | GCGGTTCGGTGCCAAAGACAGG |
| rt-mus-Sele | CTGCCAAAGCCTTCAATCGT | CTAGTAGAGGGCTGGCCTTG |
| rt-mus-Gapdh | GTGCAGTGCCAGCCTCGTCC | GCCACTGCAAATGGCAGCCC |
| rt-mus-Icam1 | GTGATGCTCAGGTATCCATCCA | CACAGTTCTCAAAGCACAGCG |
| rt-mus-Lats1 | TGGTGACTCTGGGGATAAAGAA | GGGAGTAACTCTGAATCCGAGAC |
| rt-mus-Taz | GGCCCTATCATTCACGGGAG | TCTGACCGGAATTTTCACCTGT |
| rt-mus-Vcam1 | AGTTGGGGATTCGGTTGTTCT | CCCCTCATTCCTTACCACCC |
| rt-mus-Yap 1 | ATCCCAGCACAGCAAATGCTCCAAA | TGGGGTCCGAGGGATGCTGT |

All patents, patent applications, and other publications, including GenBank Accession Numbers, cited in this application are incorporated by reference in the entirety for all purposes.

### LIST OF REFERENCES

1. Halder, G., Dupont, S. & Piccolo, S. Transduction of mechanical and cytoskeletal cues by YAP and TAZ. Nature Rev. Mol. Cell Biol. 13, 591-600 (2012).
2. Cunningham, K. S. & Gotlieb, A. I. The role of shear stress in the pathogenesis of atherosclerosis. Lab. Invest. 85, 9-23 (2005).
3. Yu, F. X., Zhao, B. & Guan, K. L. Hippo pathway in organ size control, tissue homeostasis, and cancer. Cell 163, 811-828 (2015).
4. Chaqour, B. & Goppelt-Struebe, M. Mechanical regulation of the Cyr61/CCN1 and CTGF/CCN2 proteins. FEBS J. 273, 3639-3649 (2006).
5. Yu, F. X. et al. Regulation of the Hippo-YAP pathway by G-protein-coupled receptor signaling. Cell 150, 780-791 (2012).
6. Sorrentino, G. et al. Metabolic control of YAP and TAZ by the mevalonate pathway. Nature Cell Biol. 16, 357-366 (2014).
7. Zhou, J. et al. Force-specific activation of Smad1/5 regulates vascular endothelial cell cycle progression in response to disturbed flow. Proc. Natl Acad. Sci. USA 109, 7770-7775 (2012).
8. Tzima, E., del Pozo, M. A., Shattil, S. J., Chien, S. & Schwartz, M. A. Activation of integrins in endothelial cells by fluid shear stress mediates Rho-dependent cytoskeletal alignment. EMBO J. 20, 4639-4647 (2001).
9. Ylänne, J. et al. Mutation of the cytoplasmic domain of the integrin β₃ subunit. Differential effects on cell spreading, recruitment to adhesion plaques, endocytosis, and phagocytosis. J. Biol. Chem. 270, 9550-9557 (1995).
10. Kim, C., Ye, F. & Ginsberg, M. H. Regulation of integrin activation. Annu. Rev. Cell Dev. Biol. 27, 321-345 (2011).
11. Vijayan, K. V. et al. Shear stress augments the enhanced adhesive phenotype of cells expressing the Pro33 isoform of integrin β₃. FEBS Lett. 540, 41-46 (2003).
12. Arthur, W. T., Petch, L. A. & Burridge, K. Integrin engagement suppresses RhoA activity via a c-Src-dependent mechanism. Curr. Biol. 10, 719-722 (2000).
13. Gong, H. et al. G protein subunit Gα₁₃ binds to integrin α_{IIb}β₃ and mediates integrin “outside-in” signaling. Science 327, 340-343 (2010).
14. Estevez, B., Shen, B. & Du, X. Targeting integrin and integrin signaling in treating thrombosis. Arterioscler. Thromb. Vasc. Biol. 35, 24-29 (2015).
15. Shen, B. et al. A directional switch of integrin signalling and a new anti-thrombotic strategy. Nature 503, 131-135 (2013).
16. Hoshiga, M., Alpers, C. E., Smith, L. L., Giachelli, C. M. & Schwartz, S. M. α_{ν}β₃ integrin expression in normal and atherosclerotic artery. Circ. Res. 77,1129-1135 (1995).
17. Weng, S. et al. β3 integrin deficiency promotes atherosclerosis and pulmonary inflammation in high-fat-fed, hyperlipidemic mice. Proc. Natl Acad. Sci. USA 100, 6730-6735 (2003).
18. Huang, W., Sherman, B. T. & Lempicki, R. A. Systematic and integrative analysis of large gene lists using DAVID bioinformatics resources. Nature Protocols 4, 44-57 (2009).
19. Bindea, G. et al. ClueGO: a Cytoscape plug-in to decipher functionally grouped gene ontology and pathway annotation networks. Bioinformatics 25, 1091-1093 (2009).
20. Zhao, B. et al. TEAD mediates YAP-dependent gene induction and growth control. Genes Dev. 22, 1962-1971 (2008).
21. Ricci, R. et al. Requirement of JNK2 for scavenger receptor A-mediated foam cell formation in atherogenesis. Science 306, 1558-1561 (2004).
22. Surapisitchat, J. et al. Fluid shear stress inhibits TNF-α activation of JNK but not ERK1/2 or p38 in human umbilical vein endothelial cells: inhibitory crosstalk among MAPK family members. Proc. Natl Acad. Sci. USA 98, 6476-6481 (2001).
23. Takabe, W. et al. Oscillatory shear stress induces mitochondrial superoxide production: implication of NADPH oxidase and c-Jun NH2-terminal kinase signaling. Antioxid. Redox Signal. 15, 1379-1388 (2011).
24. Zanconato, F. et al. Genome-wide association between YAP/TAZ/TEAD and AP-1 at enhancers drives oncogenic growth. Nature Cell Biol. 17, 1218-1227 (2015).
25. Liu, X. et al. Tead and AP1 coordinate transcription and motility. Cell Reports 14, 1169-1180 (2016).
26. Ma, X. et al. Impaired Hippo signaling promotes Rho1-JNK-dependent growth. Proc. Natl Acad. Sci. USA 112, 1065-1070 (2015).
27. Hoffmann, E., Dittrich-Breiholz, O., Holtmann, H. & Kracht, M. Multiple control of interleukin-8 gene expression. J. Leukoc. Biol. 72, 847-855 (2002).
28. Ran, F. A. et al. In vivo genome editing using Staphylococcus aureus Cas9. Nature 520, 186-191 (2015).
29. Wang, N. et al. Shear stress regulation of Kruppel-like factor 2 expression is flow pattern-specific. Biochem. Biophys. Res. Commun. 341, 1244-1251 (2006).
30. Miao, H. et al. Effects of flow patterns on the localization and expression of VE-cadherin at vascular endothelial cell junctions: in vivo and in vitro investigations. J. Vasc. Res. 42, 77-89 (2005).
31. Sun, X. et al. Activation of integrin α5 mediated by flow requires its translocation to membrane lipid rafts in vascular endothelial cells. Proc. Natl Acad. Sci. USA 113, 769-774 (2016).
32. Zhao, B. et al. Inactivation of YAP oncoprotein by the Hippo pathway is involved in cell contact inhibition and tissue growth control. Genes Dev. 21, 2747-2761 (2007).
33. Varelas, X. et al. TAZ controls Smad nucleocytoplasmic shuttling and regulates human embryonic stem-cell self-renewal. Nature Cell Biol. 10, 837-848 (2008).
34. Takagi, J., Petre, B. M., Walz, T. & Springer, T. A. Global conformational rearrangements in integrin extracellular domains in outside-in and inside-out signaling. Cell 110, 599-611 (2002).
35. Luo, J. et al. A protocol for rapid generation of recombinant adenoviruses using the AdEasy system. Nature Protocols 2, 1236-1247 (2007).
36. Stemmer, M., Thumberger, T., Del Sol Keyer, M., Wittbrodt, J. & Mateo, J. L. CCTop: An intuitive, flexible and reliable CRISPR/Cas9 target prediction Tool. PLoS ONE 10, e0124633 (2015).
37. Varadi, K. et al. Novel random peptide libraries displayed on AAV serotype 9 for selection of endothelial cell-directed gene transfer vectors. Gene Ther. 19, 800-809 (2012).
38. Grieger, J. C., Choi, V. W. & Samulski, R. J. Production and characterization of adeno-associated viral vectors. Nature Protocols 1,1412-1428 (2006).

1. A method for treating or preventing a cardiovascular disease or an inflammatory disease in a subject, comprising administering to the subject a composition comprising an effective amount of an inhibitor of YAP or an activator of integrin β3.

2. The method of claim 1, wherein the subject has been diagnosed with a cardiovascular disease or an inflammatory disease.

3. The method of claim 1, wherein the subject is at risk of a cardiovascular disease or an inflammatory disease but has not been diagnosed with a cardiovascular disease or an inflammatory disease.

4. The method of claim 2, wherein the composition is a medicament administered by injection or oral ingestion.

5. The method of claim 2, wherein the composition is a dietary supplement administered by oral ingestion.

6. A method for identifying a modulator of integrin-YAP/TAZ signaling pathway, comprising the steps of:
(a) placing an endothelial cell under unidirectional shear stress or under disturbed flow;
(b) contacting the cell with a candidate compound and determining YAP phosphorylation level at Ser127; and
(c) comparing the YAP phosphorylation level at Ser127 obtained in step (b) with YAP phosphorylation level at Ser127 in a control endothelial cell under unidirectional shear stress but not contacted with the candidate compound; and
(d) determining the candidate compound as an inhibitor of YAP or activator of integrin β3 when the YAP phosphorylation level at Ser127 obtained in step (b) is greater than the YAP phosphorylation level at Ser127 in the control endothelial cell, and determining the candidate compound as an activator of YAP or inhibitor of integrin β3 when the YAP phosphorylation level at Ser127 obtained in step (b) is less than the YAP phosphorylation level at Ser127 in the control endothelial cell.

7. The method of claim 6, wherein the endothelial cell is a human umbilical vein endothelial cell (HUVEC) or human aortic endothelial cell (HAEC).

8. (canceled)

9. (canceled)

10. A method for identifying a modulator of integrin-YAP/TAZ signaling pathway, comprising the steps of:
(a) placing an endothelial cell under unidirectional shear stress or under disturbed flow;
(b) contacting the cell with a candidate compound and determining integrin β3-Gβ13 association level; and
(c) comparing the integrin β3-Gα13 association level obtained in step (b) with integrin β3-Gα13 association level in a control endothelial cell under unidirectional shear stress but not contacted with the candidate compound; and
(d) determining the candidate compound as an inhibitor of YAP or activator of integrin β3 when the integrin β3-Gα13 association level obtained in step (b) is greater than the integrin β3-Gα13 association level in the control endothelial cell, and determining the candidate compound as an activator of YAP or inhibitor of integrin β3 when the integrin β3-Gα13 association level obtained in step (b) is less than the integrin β3-Gα13 association level in the control endothelial cell.

11. The method of claim 10, wherein the endothelial cell is a human umbilical vein endothelial cell (HUVEC) or human aortic endothelial cell (HAEC).

12. (canceled)

13. (canceled)

14. A kit for treating or preventing a cardiovascular disease or an inflammatory disease in a subject, comprising (1) a composition comprising an effective amount of an activator of YAP or inhibitor of integrin β3; and (2) another agent effective for treating or preventing a cardiovascular disease or an inflammatory disease.

15. The kit of claim 14, further comprising an instruction manual.
